(19) United States
(12) Patent Application Publication
(10) Pub. No.: US 2026/0240888 A1
(43) Pub. Date: Aug. 20, 2026

(54) METHODS AND PHARMACEUTICAL COMPOSITIONS FOR TREATING AGE-RELATED DISEASES

(71) Applicant: Neurxstem, Heath, OH (US)

(72) Inventor: Rene ANAND, Westerville, OH (US)

(21) Appl. No.: 19/471,187

(22) PCT Filed: Apr. 10, 2024
(86) PCT No.: PCT/US2024/023947

§ 371 Date: Sep. 30, 2025

Related U.S. Application Data
(60) Provisional application No. 63/458,421, filed on Apr. 10, 2023.

(51) Int. Cl. A61K 31/7076; A61K 31/155; A61K 31/436; A61K 31/4545; A61K 48/00; A61P 25/28; C12N 5/0793; C12N 15/113; C12Q 1/6883; C12Q 1/6886

(52) U.S. Cl. CPC A61K 31/7076; A61K 31/155; A61K 31/436; A61K 31/4545; A61P 25/28; C12N 5/0619; C12N 15/1137; C12N 15/1138; C12Q 1/6883; C12Q 1/6886; A61K 48/00; C12N 2310/11; C12N 2503/04; C12Q 2600/158

(57) ABSTRACT

Methods for using gene expression changes and mutations in neural organoids to identify and assess effectiveness of treatment methodologies and effective pharmaceutical compositions for treating a disease, disorder, or disability as a consequence of biological aging are disclosed.

### PRIORITY CLAIM

This application claims priority to U.S. provisional application No. 63/458,421, filed on Apr. 10, 2023, incorporated herein by reference in its entirety.

### FIELD OF THE INVENTION

This disclosure provides methods for reducing or ameliorating age-related disease, disorder, or disability severity. The methods provided herein include methods for early diagnostics of diseases or disabilities related to biological aging and administration of therapeutically effective amounts of pharmaceutical compositions for reducing or ameliorating age-related disease, disorder, or disability severity. Said compositions are capable of correcting or reducing dysfunctional expression of genes related to those discovered using an in vitro neural organoid platform for accelerated aging in human progeroid syndromes (including Werner Syndrome and Hutchinson-Gilford progeroid syndrome) and significant overlap of their expression in skin fibroblasts. Further provided are pharmaceutical compositions comprising one or a plurality of active pharmaceutical ingredients alone or in combination to achieve reduction or amelioration of dementia.

### BACKGROUND OF THE INVENTION

Aging and the sequalae thereof in animal organs and tissues is a universal phenomenon. In humans in particular aging is detected in all organ systems, particularly the brain. Aging is broadly defined as an irreversible and inevitable biological process, which is characterized by progressive deterioration of social, physical and mental conditions of an individual organism with advancing chronological age, which generally starts after sexual maturity and ultimately results in morbidity (Melzer et al., 2020, The genetics of human ageing. Nat. Rev. Genet. 21:88-101).

The human brain, and diseases associated with it have been the object of investigation and study by scientists for decades. Throughout this time, neurobiologists have attempted to increase their understanding of the brain's capabilities and functions. Neuroscience has typically relied on experimental manipulation of living brains or tissue samples, but a number of factors have limited scientific progress. For ethical and practical reasons, obtaining human brain tissue is difficult, while most invasive techniques are impossible to use on humans whilst they are still alive. Experiments in animals are expensive and time-consuming and many animal experiments are conducted in rodents, which have a brain structure and development that vary greatly from humans. Results obtained in animals must be verified in long and expensive human clinical trials and much of the time such animal disease models are not fully representative of disease pathology in the human brain.

Humans and other mammals are subject to a variety of forms of age-related disease, disorder, or disability and fragility characterized generally as a diminution of overall well-being or cessation of memory and functional cognition. Examples of specific age-related diseases include Hutchinson-Gilford progeroid syndrome and Werner syndrome, also collectively called progeroid syndromes (PG), a hereditary condition associated with premature aging and an increased risk of cancer and other diseases. Mutations (alterations) in certain genes, such as the LMNA gene, WRN gene and DHX9 gene are known to cause Hutchinson-Gilford progeroid syndrome and Werner syndrome.

Alzheimer's Disease (AD) is a common form of irreversible degenerative brain disorder that is associated with memory loss and interferes with other intellectual abilities that complicate daily life. Alzheimer's disease accounts for 60 to 80 percent of dementia cases. Disease onset occurs most often for individuals in their mid-60s and is estimated to affect approximately five million individuals at present. However, disease onset occurs many years prior to physical expression of symptoms. The cost to society currently exceeds $270 billion and no effective treatment currently exists.

These and other consequences of biological aging create a need in the art to develop therapeutically effective treatments for such maladies.

### SUMMARY OF THE INVENTION

This invention provides methods for reducing or ameliorating diseases and disabilities in an individual as a consequence of biological aging, including but not limited to Alzheimer's Disease, Alzheimer's Disease Related Dementia (AD/ADRD). Mixed Etiology Dementia (MED), Lewy Body dementia, fronto-temporal dementia, Progeroid syndromes, Hutchinson-Gilford progeroid syndrome, Werner syndrome, vascular contributors to dementia, or cancer, said methods comprising administering to the individual a therapeutically effective amount of a pharmaceutical composition comprising a drug capable of reducing or correcting dysfunctional expression of genes related to biological aging in vitro in a neural organoid platform that accordingly reduces or ameliorates disease severity in such individuals. Specifically, the methods provided herein rely upon identification of genes related to biological aging and specifically AD/ADRD, MED, Lewy Body dementia, fronto-temporal dementia, Progeroid syndromes, Hutchinson-Gilford progeroid syndrome, Werner syndrome, vascular contributors to dementia, or cancer that show dysfunctional or altered gene expression in neural organoid platforms in vitro for which the drugs disclosed herein are capable of reducing this dysfunctional expression. In particular embodiments these genes are set forth in Tables 3-11. In specific embodiments, the neural organoid sample is procured from minutes to hours up to 15 weeks post inducement.

A particular class of drugs provided herein are agonists of adenosine A2a receptor. (Genbank Accession No. NP_001265429.1) encoded by ADORA2A (Genbank Accession No. NM_001278497.2). In specific embodiments, such drugs include methyl (1R,4r)-4-(3-(6-amino-9-((2R,3R,4S,5S)-5-(ethylcarbamoyl)-3,4-dihydroxytetrahydrofuran-2-yl)-9H-purin-2-yl)prop-2-yn-1-yl)cyclohexane-1-carboxylate (known in the art as apadenoson). In other specific embodiments, such drugs include 2-p-(2-Carboxyethyl) phenethylamino-5′-N-ethylcarboxamidoadenosine hydrochloride hydrate (known in the art as CGS-21680). In addition to such particular and specific embodiments this disclosure encompasses related drug molecules that are agonists of adenosine A2a receptors encoded by ADORA2A and allelic variants thereof, particularly such variants associated with biological aging including but not limited to Alzheimer's Disease Related Dementia (AD/ADRD), Mixed Etiology Dementia (MED) Lewy Body dementia, fronto-temporal dementia, Progeroid syndromes, Hutchinson-Gilford progeroid syndrome, Werner syndrome, or vascular contributors to dementia. Additional drugs provided herein are methyl (1R,4r)-4-(3-(6-amino-9-((2R,3R,4S,5S)-5-(ethylcarbamoyl)-3,4-dihydroxytetrahydrofuran-2-yl)-9H-purin-2-yl)prop-2-yn-1-yl)cyclohexane-1-carboxylate (apadenoson), metformin, or rapamycin.

Another particular class of drugs provided herein are drugs that increase activity of endocannabinoid anandamide receptor encoded by CNR1 gene (Genbank Accession No. NM_016083.6). In specific embodiments, such drugs include inhibitors of Fatty Acid Amide Hydrolase such as PF-3845.

The described method uses data obtained from neural organoid which is corroborated in postmortem tissues from idiopathic patients and identifies changes in gene expression for aging including age-related dementia. In specific embodiments, the method can be used with induced pluripotent stem cells from any skin cell, tissue, or organ from the human body for diagnostics, therapeutic target discovery, and drug development. In specific embodiments, the method and/or neural organoid is used in patient specific toxicology to identify genes involved in a patient's selective vulnerability to infectious agents or environmental toxins. In specific embodiments, the method can be used to identify nutritional and toxicological care that can begin even before birth so that the child develops normally in utero. In specific embodiments, the measured genes are representative of proteins or their metabolites dysregulated in disease, disorder, or disability. In specific embodiments, the method can be used to diagnosis of aging including age-related dementia and cancers at birth.

The invention also provides pharmaceutical compositions of drugs capable of reducing or ameliorating diseases and disabilities in an individual as a consequence of biological aging, specifically including but not limited to Alzheimer's Disease, Alzheimer's Disease Related Dementia (AD/ADRD), Mixed Etiology Dementia (MED), Lewy Body dementia, fronto-temporal dementia, Progeroid syndromes, Hutchinson-Gilford progeroid syndrome, Werner syndrome, or vascular contributors to dementia. In specific embodiments the invention provides pharmaceutical compositions comprising therapeutically effective amounts of drugs capable of reducing or correcting dysfunctional expression of genes related to said diseases and disabilities. As disclosed herein, such genes are identified from a neural organoid platform wherein administration of the drugs in vitro reduces or corrects this dysfunctional gene expression toward normalcy, and accordingly ameliorates disease severity in an individual having diseases and disabilities in an individual as a consequence of biological aging when the pharmaceutical composition is administered to such patients. In particular embodiments these genes are set forth in Tables 3-11.

In specific embodiments the pharmaceutical compositions comprise drugs that are agonists of adenosine A2a receptor. (Genbank Accession No. NP_001265429.1) encoded by ADORA2A (Genbank Accession No. NM_001278497.2). In specific embodiments, such drugs include methyl (1R,4r)-4-(3-(6-amino-9-((2R,3R,4S,5S)-5-(ethylcarbamoyl)-3,4-dihydroxytetrahydrofuran-2-yl)-9H-purin-2-yl)prop-2-yn-1-yl)cyclohexane-1-carboxylate (known in the art as apadenoson). In other specific embodiments, such drugs include 2-p-(2-Carboxyethyl) phenethylamino-5′-N-ethylcarboxamidoadenosine hydrochloride hydrate (known in the art as CGS-21680). In addition to such particular and specific embodiments this disclosure encompasses related drug molecules that are agonists of adenosine A2a receptors encoded by ADORA2A and allelic variants thereof, particularly such variants associated with diseases and disabilities in an individual as a consequence of biological aging including Alzheimer's Disease, Alzheimer's Disease Related Dementia (ADRD), Mixed Etiology Dementia (MED), Lewy Body dementia, fronto-temporal dementia, Progeroid syndromes, Hutchinson-Gilford progeroid syndrome, Werner syndrome, or vascular contributors to dementia. Additional drugs provided herein are methyl (1R,4r)-4-(3-(6-amino-9-((2R,3R,4S,5S)-5-(ethylcarbamoyl)-3,4-dihydroxytetrahydrofuran-2-yl)-9H-purin-2-yl)prop-2-yn-1-yl)cyclohexane-1-carboxylate (apadenoson), metformin, or rapamycin.

In specific embodiments the pharmaceutical compositions comprise drugs that increase activity of endocannabinoid anandamide receptor encoded by CNR1 gene (Genbank Accession No. NM_016083.6). In specific embodiments, such drugs include inhibitors of Fatty Acid Amide Hydrolase such as PF-3845.

The invention also provides methods for performing gene therapy in a cell to correct expression of one or a plurality of adenosine receptor network components identified as ADORA1, ADORA2A, ADORA2B, CNR1, and EZH2 genes and their modulators resulting in modification of gene expression in the one or a plurality of components. In specific embodiments, the gene therapy is performed using long non-coding RNA species.

In specific embodiments, the gene therapy is performed using an anti-sense oligonucleotide (ASO) to modify expression of the adenosine receptor network components identified as ADORA1, ADORA2A, ADORA2B, CNR1 and EZH2. In this method, the effects of the gene therapy can be measured by detecting changes in expression of genes encoding human LMNA, WRN, DHX9, A2M, PSEN2, ApoE, APP variants or sporadic AD. These genes are one or a plurality of genes identified in Tables 3-11. In specific embodiments, the gene therapy is performed using an anti-sense oligonucleotide (ASO) to correct expression of the human LMNA, WRN, DHX9, A2M, PSEN2, ApoE, APP variants or sporadic AD.

The invention also provides method for treating aging including age-related dementia and cancers in a human, using a patient-specific pharmacotherapy, the method comprising:
a) procuring one or a plurality of cell samples from a patient, comprising one or a plurality of cell types including but not limited to fibroblasts, buccal cavity cells, or peripheral blood monocytes;
b) detecting genes from the procured one or a plurality of cell samples that are differentially expressed, wherein these genes are also differentially expressed in humans with accelerated aging including age-related dementia and cancers;
c) performing assays on the patient specific neural organoid to identify therapeutic agents that alter the differentially expressed during aging including age-related dementia and cancers genes in the patient-specific neural organoid sample; and
d) administering a therapeutic agent for aging including age-related dementia and cancers to treat the human

In specific embodiments, the fibroblast is used as a developmental tool for predicting the risk of aging including age-related dementia and cancers in a newborn, or used as a drug discovery tool, or used to follow the onset, progression, and/or treatment of aging including age-related dementia and cancers.

These and other data findings, features, and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE FIGURES

FIG. 1A is a diagram setting forth the rationale for producing a neural organoid platform (NNOP) as used herein shown in a micrograph of a 4× dark field image of a NNOP.

FIG. 1B is a photomicrograph of a NNOP illustrated by comparison with a diagram of a 5-week-old fetus and corresponding anatomical structures.

FIG. 2 is a diagram illustrating the strategy for identifying gene expression and metabolic profiles in NNOP related to Alzheimer's Disease Related Dementia (ADRD), Mixed Etiology Dementia (MED), Lewy Body dementia, fronto-temporal dementia, Progeroid syndromes, Hutchinson-Gilford progeroid syndrome, Werner syndrome, vascular contributors to dementia, or cancer for use in drug development.

FIG. 3 shows results of transcriptomic data produced using NNOP from familial amyloid plaque disorders (APP) and sporadic (SPOR) donor. Each dot is an RNA expression level of each gene. Each X and Y axis shows RPKM (Reads Per Kilo base per Million reads) which represents a gene expression level of each gene from two randomly selected organoid out of hundreds of organoids. Independent NNOP sample transcriptomic data are shown at different time points in development. Plots represent high data reproducibility for ~15,000 genes expressed at 12 weeks in culture (W12) with a variance of <0.95 in independent replicates.

FIG. 4 shows ¹H NMR metabolomic results to track changes in metabolites of physiological significance in ADRD/MED-APP-NNOP. Independent (bottom; blue; organoid #1) and (top; red; organoid #2) samples traces of ADRD/MED-APP-NNOP show high reproducibility (peak area replicability), and thus demonstrated feasibility for tracking changes in metabolites of interest in age-related changes including AD/ADRD in response to any therapeutic drug.

FIG. 5 is a diagram showing the strategy to identify biomarkers of aging-related diseases with pProgeroid syndromes as a model for aging. Clinical validation of NNOP for AD/ADRD is apparent from significant corroboration of AD-APP-NNOP transcriptomic data with clinical postmortem AD brain transcriptomic data analyses (Annese et al. Sci Rep. 2018; 8(1):4282) of a match with a statistical p-value of 1e⁻¹⁰³ of a match by chance alone. The biomarker genes in common between AD-NNOP and postmortem AD brain also show significant overlap with transcriptomic data from accelerated aging Hutchinson-Gilford progeroid syndrome patient sample-derived PG-NNOP including expression of genes such as WRN and DHX9. These overlapped biomarkers of AD-NNOP, postmortem AD brain, and PG-NNOP can be used as early diagnostic biomarkers and drug targets.

FIG. 6 shows normal-NNOP and PG-NNOP having highly replicable, reliable, robust transcriptomic data. Each dot is an RNA expression level for each gene. Each X and Y axis shows RPKM (Reads Per Kilo base per Million reads) which represents a gene expression level of each gene from two randomly selected organoids out of hundreds of organoids. Independent NNOP sample transcriptomic data at week 12 in development. Plots represent high data reproducibility for ~13,000 genes expressed with a variance of <0.95 in independent replicates.

FIG. 7 shows Normal-NNOP express all brain regions and all cell types including microglia. FIG. 7A shows the anatomy of the whole organoid with corresponding brain structures. The Tables show Whole Genome Developmental Transcriptomic, or gene expression profile that is signature of the indicated brain region. The numbers indicated in the table are normalized gene expression at Week 1 (W1), 4, and 12 in culture for neural organoids obtained from a healthy White Caucasian Female (WCF). The numbers in darkest gray in grayscale view (or red in color view) correspond to the highest expression (the yellow and green highlight in color view (or light gray and medium gray in grayscale view) depict medium and low expression respectively). FIG. 7B is 3D Clarity Immuno-fluorescent staining and Confocal Imaging showing expression of the biomarker protein of the indicated region on the neural organoid.

### DETAILED DESCRIPTION

Unless defined otherwise, all technical and scientific terms used herein have the same meaning as is commonly understood by one of ordinary skill in the art. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). These references are intended to be exemplary and illustrative and not limiting as to the source of information known to the worker of ordinary skill in this art. As used herein, the following terms have the meanings ascribed to them below, unless specified otherwise.

It is noted here that as used in this specification and the appended claims, the singular forms “a,” “an,” and “the” also include plural reference, unless the context clarity dictates otherwise.

The term “about” or “approximately” means within 25%, such as within 20% (or 5% or less) of a given value or range.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention, it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the term “neural organoid” and “neural organoid platform” mean a non-naturally occurring three-dimensional organized cell mass that is cultured in vitro from a human induced pluripotent stem cell and develops similarly to the human nervous system in terms of neural marker expression and structure. Further a neural organoid has two or more regions. The first region expresses cortical or retinal marker or markers. The remaining regions each express markers of the brain stem, cerebellum, and/or spinal cord. Neural organoids falling within this definition herein include organoids as disclosed in U.S. Pat. No. 11,345,890, incorporated herein in its entirety.

As used herein, by “neural marker” is meant any protein or polynucleotide, the expression of which is associated with a neural cell fate. Neural markers are any protein or polynucleotide expressed consistent with a cell lineage. Exemplary neural markers include markers associated with the hindbrain, midbrain, forebrain, or spinal cord. One skilled in the art will understand that neural markers are representative of the cerebrum, cerebellum and brainstem regions. Exemplary brain structures that express neural markers include the cortex, hyopthalamus, thalamus, retina, medulla, pons, and lateral ventricles. Further, one skilled in the art will recognize that within the brain regions and structures, granular neurons, dopaminergic neurons, GABAergic neurons, cholinergic neurons, glutamatergic neurons, serotonergic neurons, dendrites, axons, neurons, neuronal, cilia, purkinje fibers, pyramidal cells, spindle cells, express neuronal markers. One skilled in the art will recognize that this list is not exhaustive or all-encompassing and that neural markers are found throughout the central nervous system including other brain regions, structures, and cell types.

As set forth herein gene expression markers for diseases and disabilities in an individual as a consequence of biological aging, specifically including but not limited to Alzheimer's Disease, Alzheimer's Disease Related Dementia (ADRD), Mixed Etiology Dementia (MED), Lewy Body dementia, fronto-temporal dementia, Progeroid syndromes, Hutchinson-Gilford progeroid syndrome, Werner syndrome, vascular contributors to dementia, or cancer are set forth in Tables 3-11 herein.

### Neural Organoids as In Vitro Models to Study Aging

Neural organoids are generated in vitro from patient tissue samples. Neural organoids were previously disclosed in U.S. Pat. No. 11,345,890, incorporated herein in its entirety. A variety of tissues can be used including skin cells, hematopoietic cells, or peripheral blood mononuclear cells (PBMCs) or in vivo stem cells directly. One of the skills in the art will further recognize that other tissue samples can be used to generate neural organoids. Use of neural organoids permits study of neural development in vitro. In one embodiment skin cells are collected in a petri dish and induced to an embryonic-like pluripotent stem cell (iPSC) that have high levels of developmental plasticity. iPSCs are grown into neural organoids in said culture under appropriate conditions as set forth herein and the resulting neural organoids closely resemble developmental patterns similar to human brain. In particular, neural organoids develop anatomical features of the retina, forebrain, midbrain, hindbrain, and spinal cord. Importantly, neural organoids express >98% of the about 15,000 transcripts found in the adult human brain. iPSCs can be derived from the skin or blood cells of humans identified with aging-related diseases such as Progeroid syndromes, Alzheimer's, or dementia.

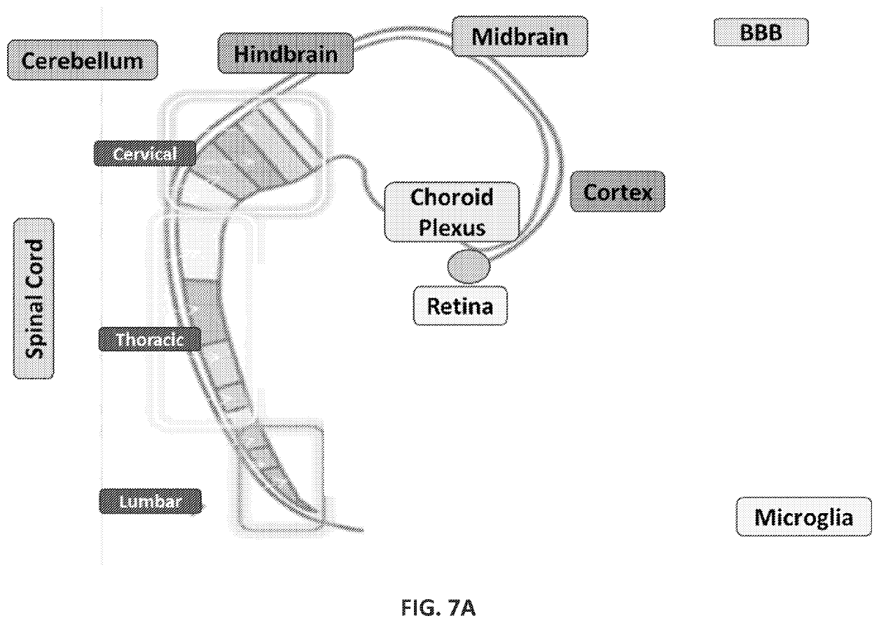

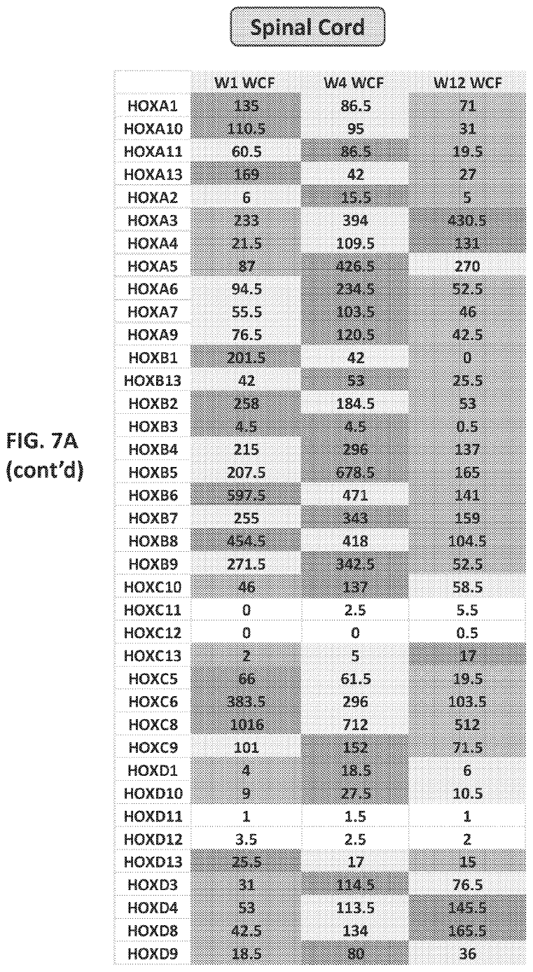

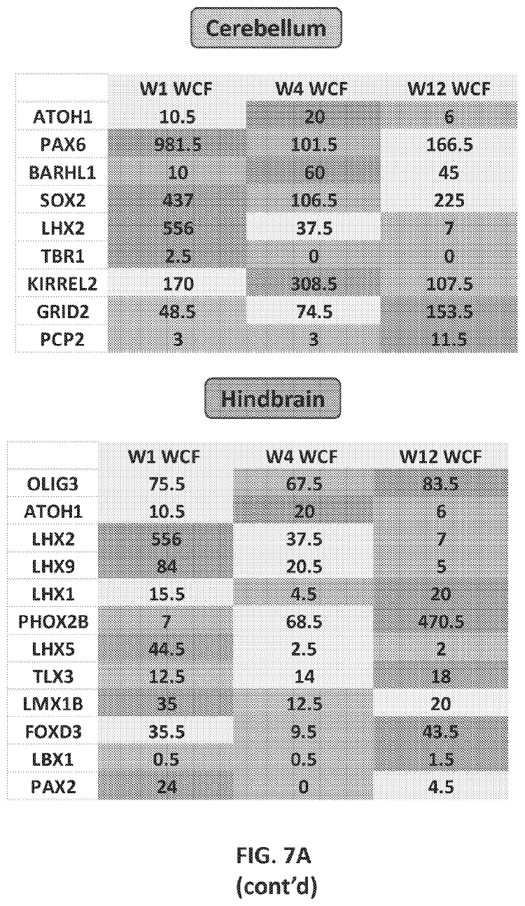

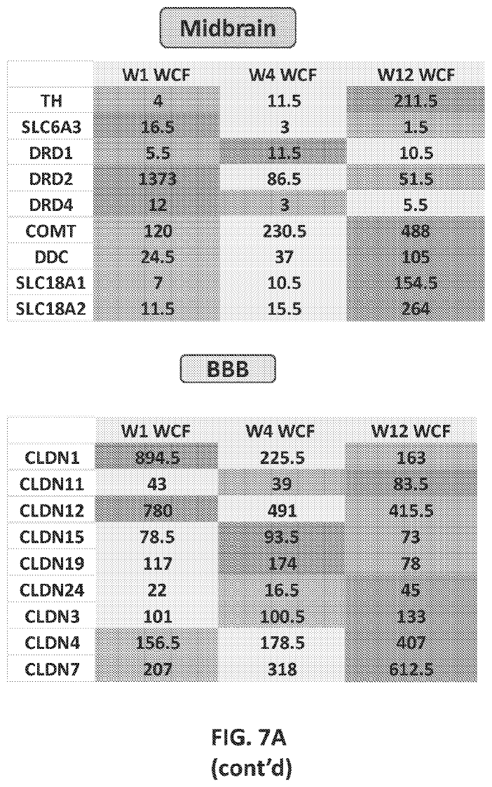

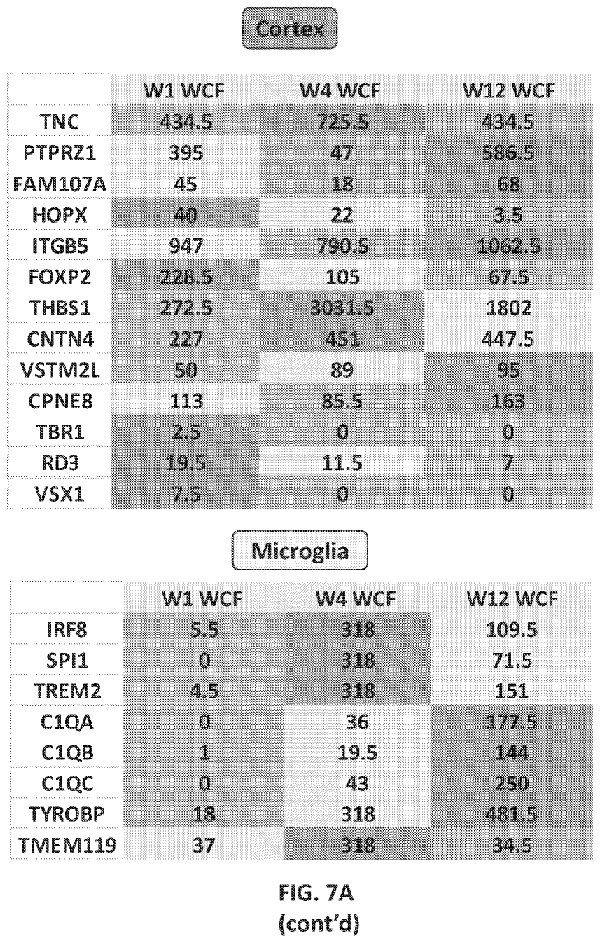

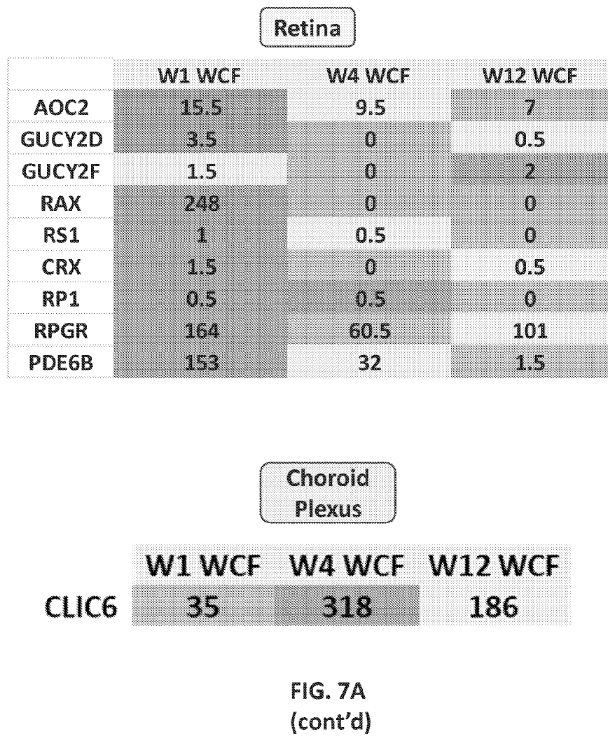

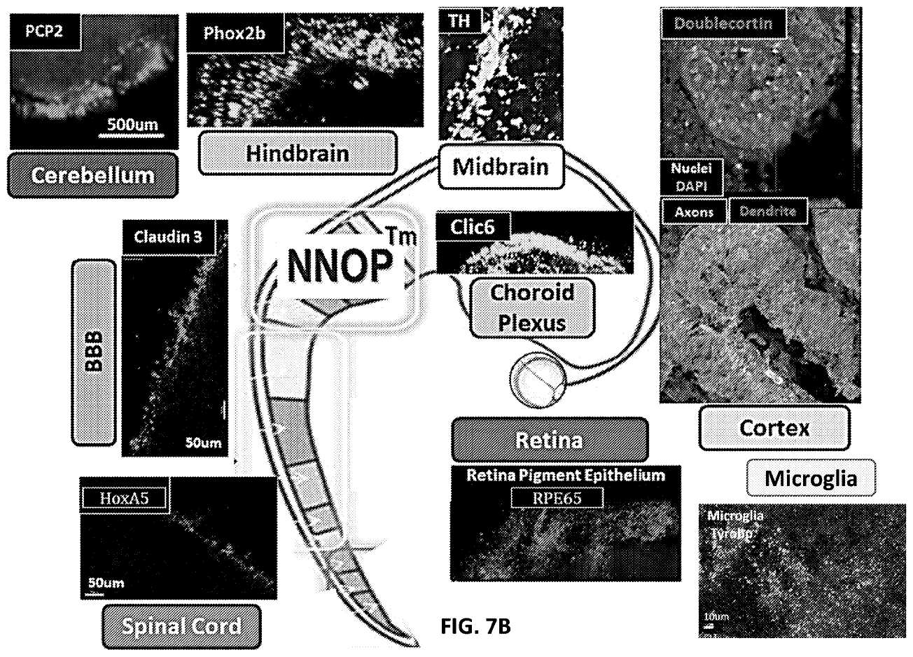

In one embodiment, the about 12-week old iPSC-derived human neural organoid has ventricles and other anatomical features characteristic of a 35-40 day old neonate. In an additional embodiment the about 12 week old neural organoid expresses beta 3-tubulin, a marker of axons as well as somato-dendritic Puncta staining for MAP2, consistent with dendrites. In yet another embodiment, at about 12 weeks the neural organoid displays laminar organization of cortical structures. Cells within the laminar structure stain positive for doublecortin (cortical neuron cytosol), Beta3 tubulin (axons) and nuclear staining. The neural organoid, by 12 weeks, also displays dopaminergic neurons and astrocytes. The neural organoid generated by the method disclosed in U.S. Pat. No. 11,345,890 has similar anatomy to typical brain structure and gene expression profile that corresponds to the various regions and cell types of the brain, namely cerebellum, hindbrain, midbrain, cortex, blood brain barrier, spinal cord, retina, choroid plexus, and microglia (FIG. 7A and FIG. 7B). These brain regions and gene expression patterns are missing in other models of neural organoids described elsewhere; and thus, making the neural organoids described herein an advantageous model that closely mimics the physiological nervous system.

The neural organoids as described here offer many advantages in studying aging diseases. The term “aging” refers to a time-related and progressive changes in an organism that lead to senescence or a decline of biological functions and of the organism's ability to adapt to metabolic stress. Aging can occur to one or multiple organs and display comorbidities including, but are not limited to, osteoarthritis, inflammatory bowel disease, pain, migraine, sarcopenia (muscle weakness), cardiac hypertrophy, and others as shown in Table 10 under clinical features.

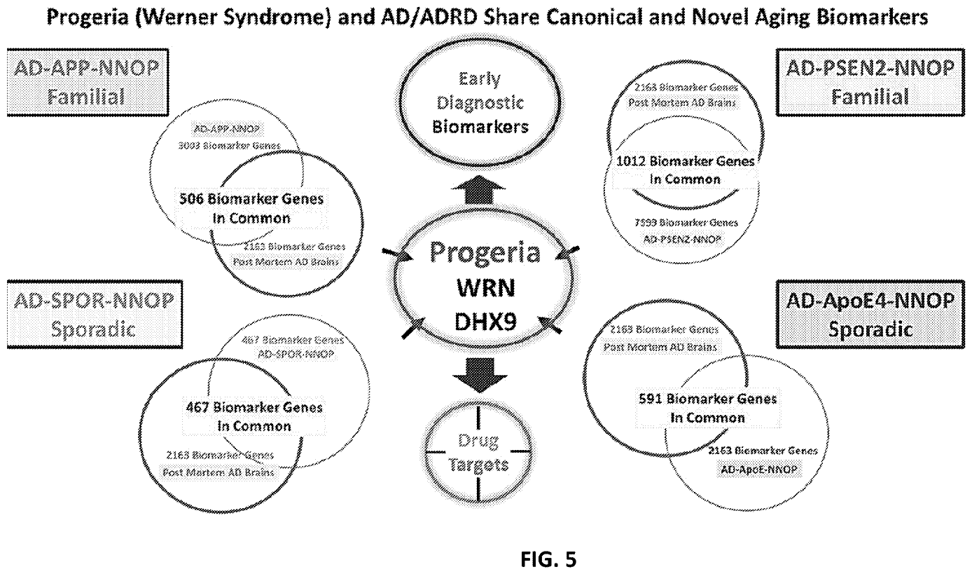

In this disclosure, Progeroid syndromes (PG) and Alzheimer's disease (AD) are used as aging-related disease models to study the neural organoids as a tool to identify clinically relevant biomakers of aging and drugs that can target these markers. Both diseases exhibit clinical features that are prominent in aging. Progeroid syndromes is a genetic accelerated aging disorder, characterized by the dramatic, rapid appearance of features associated with normal aging. Several common types of Progeroid syndromes include Hutchinson-Gilford progeroid syndrome, Werner syndrome, Bloom syndrome, Cockayne syndrome and others. Some genes have been found to be mutated and/or differentially expressed in these syndromes including WRN, LMNA, DHX9, and others. Meanwhile, Alzheimer's disease is a common type of dementia and is considered a pre-mature aging disease of the brain. Strikingly, the transcriptomic data from the neural organoids obtained from AD patients (AD-NNOP) statistically overlap with the transcriptomic data from postmortem brain samples of AD patients (FIG. 5). Furthermore, these overlapped biomarkers are also dysregulated in neural organoids obtained from PG patients (PG-NNOP), demonstrating that there are common pathways and gene expressions that are altered in aging-related diseases (Table 5A and Table 5B). Treatment with drugs on AD-NNOP or PG-NNOP rescues expression of these dysregulated genes further suggesting that these biomarkers are relevant in progression of the diseases (Tables 6-9).

The neural organoids are powerful tools to study aging in vitro. The neural organoid offers the advantages of replicability, reliability and robustness, as shown herein using replicate neural organoids from the same source of iPSCs. In one aspect at least one cell sample reprogrammed to the induced pluripotent stem cell is a fibroblast derived from skin or blood cells from humans.

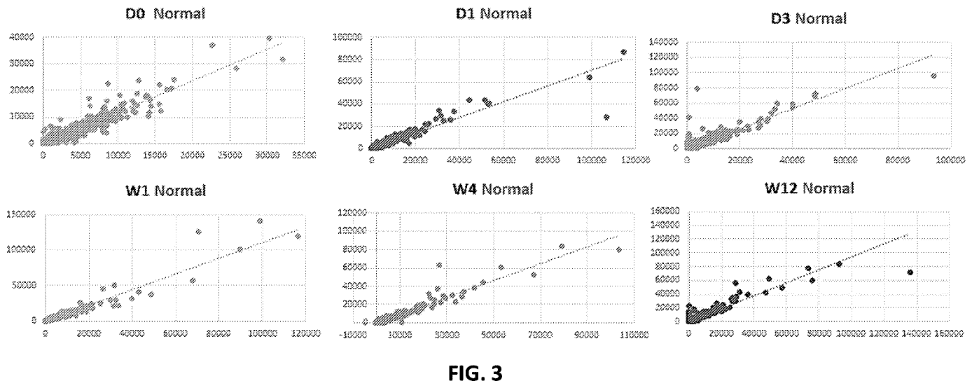

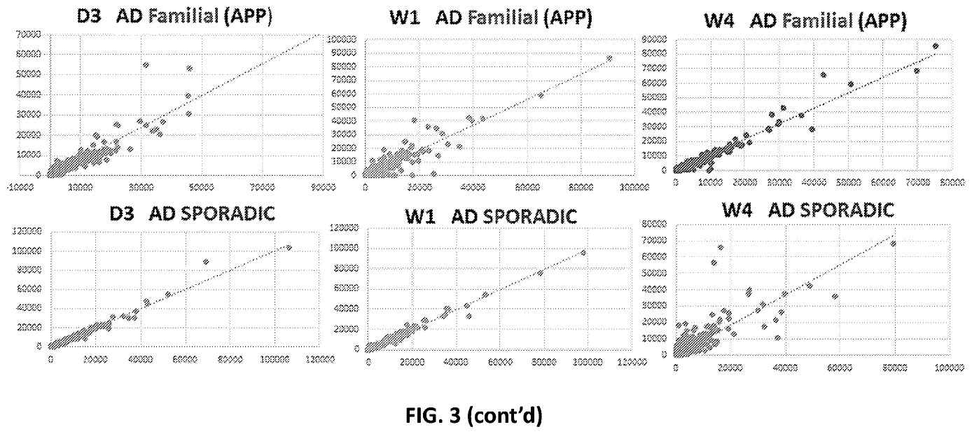

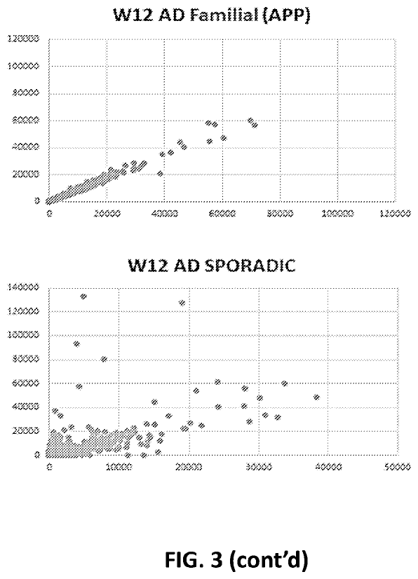

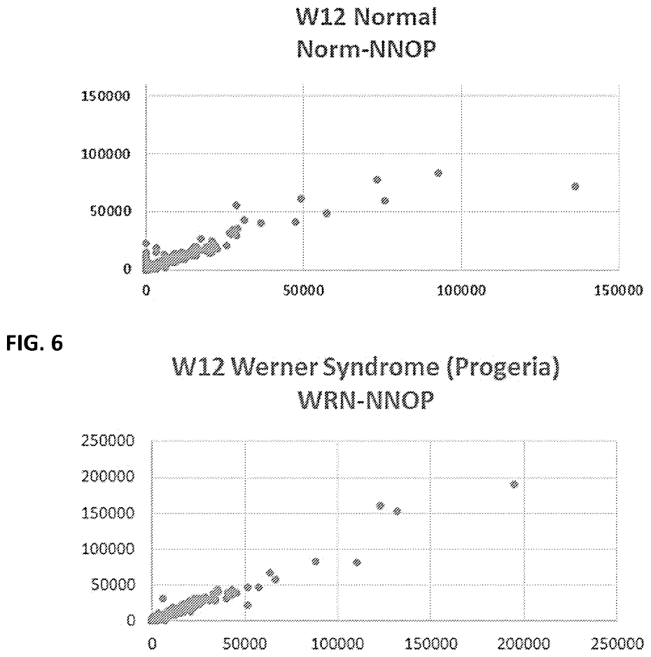

Firstly, the neural organoids obtained from the same patient's sample (either from skin or blood sample) are highly uniform and replicable in regards to the gene expression. FIG. 3 and FIG. 6 show that expression of one gene in two independent neural organoids randomly selected from thousands of organoids that are generated from one patient's sample, are closely matched with an overall variance for all the genes tested of over 0.95. The transcriptomic data generated from the NNOP are reliable and robust because they corroborate the transcriptomic data obtained from postmortem samples (FIG. 5). Additionally, compared to expensive, labor intensive, and time-consuming animal models, neural organoids can be generated from relatively easy-to-obtain skin or blood samples in live patients. Strikingly, Table 10 shows that the gene expression profile from AD patient's fibroblasts overlap with the gene expression of PG-NNOP. Some of the gene expression also correlate with some clinical features of aging. The data shows that aging biomarkers appear similarly across aging organs and can be measured to assess some clinical aging symptoms. The clinically relevant biomarkers of neural organoids can potentially be used as early diagnostic tools to identify individual in risks of developing an aging-related diseases or identify treatments in patients at early stage of the disease when the symptoms are not yet to arise.

### Developmental Transcriptomics

A “transcriptome” is a collection of all RNA, including messenger RNA (mRNA), long non-coding RNAs (lncRNA), microRNAs (miRNA) and, small nucleolar RNA snoRNA), other regulatory polynucleotides, and regulatory RNA (lncRNA, miRNA) molecules expressed from the genome of an organism through transcription therefrom. Thus, transcriptomics is the study of the mRNA transcripts produced by the genome at a given time in any particular cell or tissue of the organism. Transcriptomics employs high-throughput techniques to analyze genome expression changes associated with development or disease. In certain embodiments, transcriptomic studies can be used to compare normal, healthy tissues and diseased tissue gene expression. In further embodiments, mutated genes or variants associated with disease or the environment can be identified.

Consistent with this, the aim of developmental transcriptomics is identifying genes associated with, or significant in, organismal development and disease and dysfunctions associated with development. During development, genes undergo up- and down-regulation as the organism develops. Thus, transcriptomics provides insight into cellular processes, and the biology of the organism.

Generally, in one embodiment RNA is sampled from the neural organoid described herein within at about one week, about four weeks, or about twelve weeks of development; most particularly RNA from all three time periods are samples. However, RNA from the neural organoid can be harvested at minutes, hours, days, or weeks after reprogramming. For instance, RNA can be harvested at about 10 minutes, 20 minutes, 30 minutes, 40 minutes, 50 minutes, and 60 minutes. In a further embodiment the RNA can be harvested 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours. In a further embodiment the RNA can be harvested at 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, or 1 week, 2 weeks, 3 weeks, 4 weeks, 5 weeks, 6 weeks, 7 weeks, 8 weeks, 9 weeks 10 weeks, 11 weeks, 12 weeks or more in culture. After enriching for RNA sequences, an expressed sequence tag (EST) library is generated and quantitated using the AmpliSeq™ technique from ThermoFisher. Exemplars of alternate technologies include RNASeq and chip-based hybridization methods. Transcript abundance in such experiments is compared in control neural organoids from healthy individuals vs. neural organoids generated from individuals with disease and the fold change in gene expression calculated and reported.

Furthermore, in one embodiment RNA from neural organoids for Alzheimer's disease, are converted to DNA libraries and then the representative DNA libraries are sequenced using exon-specific primers for 20,814 genes using the AmpliSeq™ technique available commercially from ThermoFisher. Reads in counts per minute (cpm)<1 are considered background noise. All cpm data are normalized data and the reads are a direct representation of the abundance of the RNA for each gene. In another informative embodiment, RNA from neural organoids for Progeroid syndromes were converted to DNA libraries and then sequenced according to this protocol.

Briefly, in one embodiment, the array consists of one or a plurality of genes identified in association with biological aging, specifically said aging related to Progeroid syndromes. In an alternative embodiment, the array consists of one or a plurality of genes used to predict risk of aging such as Alzheimer's disease. In a further alternative embodiment, reads contain a plurality of genes that are used to treat aging-related disease in a human, using patient-specific pharmacotherapy known to be associated with aging. In one aspect, the gene libraries can be comprised of disease-specific gene as provided in Tables 3-11 or a combination of genes in Tables 3-11 with alternative disease specific genes. Exemplarily, changes in expression or mutation of disease-specific genes are detected using such sequencing, and differential gene expression detected thereby, qualitatively by detecting a pattern of gene expression or quantitatively by detecting the amount or extent of expression of one or a plurality of disease-specific genes or mutations thereof. Results of said assays using the AmpliSeq™ technique can be used to identify genes that can predict disease risk or onset and can be targets of therapeutic intervention. In further embodiments, hybridization assays can be used, including but not limited to sandwich hybridization assays, competitive hybridization assays, hybridization-ligation assays, dual ligation hybridization assays, or nuclease assays.

### Neural Organoids and Pharmaceutical Testing

Neural organoids are useful for pharmaceutical testing. Currently, drug screening studies including toxicity, safety and or pharmaceutical efficacy, are performed using a combination of in vitro work, rodent/primate studies and computer modeling. Collectively, these studies seek to model human responses, in particular physiological responses of the central nervous system.

Human neural organoids are advantageous over current pharmaceutical testing methods for several reasons. First neural organoids are easily derived from healthy and diseased patients, mitigating the need to conduct expensive clinical trials. Second, rodent models of human disease are unable to mimic physiological nuances unique to human growth and development. Third, use of primates creates ethical concerns. Finally, current methods are indirect indices of drug safety. Alternatively, neural organoids offer an inexpensive, easily accessible model of human brain development. This model permits direct, and thus more thorough, understanding of the safety, efficacy, and toxicity of pharmaceutical compounds.

Starting material for neural organoids is easily obtained from healthy and diseased patients. Further, because human organoids are easily grown they can be produced en mass. This permits efficient screening of pharmaceutical compounds.

Neural organoids are advantageous for identifying biomarkers of a disease or a condition, the method comprising a) obtaining a biological sample from a human patient; and b) detecting whether at least one biomarker is present in the biological sample by contacting the biological sample with an array comprising binding molecules specific for the biomarkers and detecting binding between the at least one biomarker and the specific binding molecules. In further embodiments, the biomarker serves as a gene therapy target.

### Relevance for Adenosine Receptors in Aging and Dementia Etiologies

Certain studies in the prior art suggest the possibility of a role (heretofore undefined and unexplored in the art) for adenosine receptors in the progression of the neuropathological changes that are observed in AD (Kinney et al., 2018, Alzheimer's & Dementia: Translational Research & Clinical Interventions, Alzheimer's and Dementia: Translational Research and Clinical Intervention 4: 575-590). For example, an association between a polymorphism of the ADORA2A gene with hippocampal volume in mild cognitive impairment and AD has been reported (Horgusluoglu-Moloch et al., 2017, Targeted neurogenesis pathway-based gene analysis identifies ADORA2A associated with hippocampal volume in mild cognitive impairment and Alzheimer's disease, Neurobiol. Aging 60: 92-103). Adenosine receptors have different functions, and the A_{2A} receptor has been more widely recognized to be associated with a broader anti-inflammatory effect throughout the body (Hasko and Pather, 2008, A_{2A} receptors in inflammation and injury: lessons learned from transgenic animals, J. Leucocyte Biol. 83: 447-455). Both receptors also regulate the release of dopamine and glutamate in the brain (Sun and Hwang, 2016, Adenosine A2B Receptor: From Cell Biology to Human Diseases, Front. Chem. 24: 37; Fuxe et al., 2007, Adenosine receptor-dopamine receptor interactions in the basal ganglia and their relevance for brain function, Physiol Behav 92:210-7; Schiffmann et al., 2007, Adenosine A2A receptors and basal ganglia physiology, Prog Neurobiol 83: 277-92; and Cunha et al., 2008, How does adenosine control neuronal dysfunction and neurodegeneration?, J. Neurobiol. 139:1019-1055) without any established etiology of neurological diseases. Set forth herein for the first time is evidence for a more general role for adenosine receptors in biological aging.

The Adenosine A_{2A} Receptor (ADORA2A) gene is expressed at a lower level compared to normal (p<0.05; Table 3, 4, and 11) in gene expression analyses using NNOP from individuals with AD/ADRD and shared biomarkers with Progeroid syndromes. Furthermore, multiple genes in the STRING analysis (ADORA1; ACTN1, ACTN3, CALM3; GRM5, NECAB2, SYNGR1 and FURIN) show epigenetic co-regulation with the ADORA2A gene. These data strongly suggest that A_{2A} agonists could augment ADORA2A anti-inflammatory pathway function. Multiple agonists have been characterized (CGS21680; DPMA; HE-NECA; ATL-146e; and CVT-3146). As set forth herein this invention utilizes two of them, CGS-21680 and Apadenoson (ATL-146e), as therapeutic compositions for treating ADRD/MED.

This invention provides methods for reducing or ameliorating disease severity in an individual having diseases and disabilities in an individual as a consequence of biological aging including but not limited to Alzheimer's Disease, Alzheimer's Disease Related Dementia (ADRD), Mixed Etiology Dementia (MED), Lewy Body dementia, fronto-temporal dementia, Progeroid syndromes, Hutchinson-Gilford progeroid syndrome, Werner syndrome, vascular contributors to dementia, or cancer comprising administering to the individual a therapeutically effective amount of a pharmaceutical composition comprising a drug capable of reducing or correcting dysfunctional expression of genes associated with diseases and disabilities in an individual as a consequence of biological aging in vitro in a neural organoid platform and accordingly reduces or ameliorates disease severity in such individuals. Specifically, the methods provided herein rely upon identification of genes associated with diseases and disabilities in an individual as a consequence of biological aging that show dysfunctional or altered gene expression in neural organoid platforms in vitro for which the drugs disclosed herein are capable of reducing this dysfunctional expression. In particular embodiments these genes are set forth in Tables 3-11.

As will be understood by the skilled worker, the majority of dementia cases in the elderly (65 years of age and older) are so-called “mixed” dementias, mainly Alzheimer's disease concomitant with cerebrovascular disease and/or Lewy body formation (see, James et al., 2012, Dementia From Alzheimer Disease and Mixed Pathologies in the Oldest Old, JAMA 307: 1798-1800; Schneider et al., 2007, Neurology 69: 2197-2204).

As used herein, the term “dementia” is intended to encompass illness particularly in humans having symptoms including diminution or cessation of memory and functional cognition. Examples of specific diseases characterized as dementia include Alzheimer's Disease (AD), which is a common form of irreversible degenerative brain disorder that is associated with memory loss and interferes with other intellectual abilities that complicate daily life. Also included in the meaning of the term dementia as used herein are Alzheimer's Disease Related Dementia (ADRD), Mixed Etiology Dementia (MED), Lewy Body dementia, fronto-temporal dementia, Progeroid syndromes, Hutchinson-Gilford progeroid syndrome, Werner syndrome, or vascular contributors to dementia.

As used herein, the terms “reducing or ameliorating” are intended to be understood to include improvements or reduction in impairments or progression thereof of symptoms of diseases and disabilities in an individual as a consequence of biological aging, particularly cognitive symptoms, of dementia, specifically including but not limited to Alzheimer's Disease, Alzheimer's Disease Related Dementia (ADRD), Mixed Etiology Dementia (MED), Lewy Body dementia, fronto-temporal dementia, Progeroid syndromes, Hutchinson-Gilford progeroid syndrome, Werner syndrome, vascular contributors to dementia, or cancer.

As used herein, the term “therapeutically effective amount” of a drug for treating diseases and disabilities in an individual as a consequence of biological aging including but not limited to dementia, specifically including but not limited to Alzheimer's Disease, Alzheimer's Disease Related Dementia (ADRD), Mixed Etiology Dementia (MED), Lewy Body dementia, fronto-temporal dementia, Progeroid syndromes, Hutchinson-Gilford progeroid syndrome, Werner syndrome, vascular contributors to dementia, or cancer will be understood to include dosage amounts that provide improvements or reduction in impairments or progression thereof of symptoms, particularly cognitive symptoms, of dementia, specifically including but not limited to Alzheimer's Disease, Alzheimer's Disease Related Dementia (ADRD), Mixed Etiology Dementia (MED), Lewy Body dementia, fronto-temporal dementia, Progeroid syndromes, Hutchinson-Gilford progeroid syndrome, Werner syndrome, vascular contributors to dementia, or cancer.

As used herein, the term “dysfunctional gene expression” will be understood to mean differences in gene expression in NNOP as shown herein produced from skin cells, inter alia by methods for producing induced pluripotent stem cells that are then differentiated into NOP s set forth in U.S. Pat. No. 11,345,890, incorporated herein in its entirety, between individuals having diseases and disabilities in an individual as a consequence of biological aging specifically including but not limited to Progeroid syndromes and Alzheimer's Disease, Alzheimer's Disease Related Dementia (ADRD) or Mixed Etiology Dementia (MED) or a genetic propensity for developing Alzheimer's Disease (AD) or Alzheimer's Disease Related Dementia (ADRD) or Mixed Etiology Dementia (MED) and individuals without Alzheimer's Disease Related Dementia (ADRD) or Mixed Etiology Dementia (MED) or a genetic propensity for developing Alzheimer's Disease Related Dementia (ADRD) or Mixed Etiology Dementia (MED) or cancer. Genetically, AD is divided into familial cases and sporadic cases which can be found in patients clinically. The familial form is due to mutations in three major genes: amyloid precursor protein (APP) gene, apolipoprotein E (ApoE), or presenilin 2 gene (PSEN2). Useful NNOPs. Useful NNOPs as set forth herein can be generated as disclosed herein from adult skin cells of individuals having genetic traits including those of sporadic Alzheimer's disease (identified as SPOR herein), amyloid plaque disorders (APP), mutations in ApoE (ApoE), or presenilin (PSEN2). The Progeroid syndromes iPSC used to bioengineer PG-NNOP was derived from fibroblast from a white, female, 14-year-old patient donor with a LMNA gene mutation with Hutchinson-Gilford progeroid Syndrome (Cat #AG27221; Coriell Biorepository, NJ).

As used herein, the term “reducing or correcting dysfunctional gene expression” refers to changes in expression of certain genes (set forth herein in Tables 3-11) in NOP in response to addition of therapeutically effective amounts of a drug a disclosed herein, wherein dysfunctional gene expression has the meaning set forth above, and degree of effect on the NOP will be understood to reduce the extent of the dysfunction in expression of said genes.

In particular embodiments, the genes having differential dysfunctional gene expression related to or associated with aging-related diseases such as Alzheimer's Disease Related Dementia (ADRD) or Mixed Etiology Dementia (MED), Lewy Body dementia, fronto-temporal dementia, Progeroid syndromes, Hutchinson-Gilford progeroid syndrome, Werner syndrome, vascular contributors to dementia, or cancer are one or more of the genes set forth herein in Tables 3-11.

A particular class of drugs provided herein are agonists of adenosine A2a receptor. (GENBANK ACCESSION NO. NP_001265429.1) encoded by ADORA2A (GENBANK ACCESSION NO. NM_001278497.2). In specific embodiments, such drugs include methyl (1R,4r)-4-(3-(6-amino-9-((2R,3R,4S,5S)-5-(ethylcarbamoyl)-3,4-dihydroxytetrahydrofuran-2-yl)-9H-purin-2-yl)prop-2-yn-1-yl)cyclohexane-1-carboxylate (known in the art as apadenoson). In other specific embodiments, such drugs include 2-p-(2-Carboxyethyl) phenethylamino-5′-N-ethylcarboxamidoadenosine hydrochloride hydrate (known in the art as CGS-21680). In addition to such particular and specific embodiments this disclosure encompasses related drug molecules that are agonists of adenosine A2a receptors encoded by ADORA2A and allelic variants thereof, particularly such variants associated with diseases and disabilities in an individual as a consequence of biological aging including but not limited to Alzheimer's Disease Related Dementia (ADRD), Mixed Etiology Dementia (MED), Lewy Body dementia, fronto-temporal dementia, Progeroid syndromes, Hutchinson-Gilford progeroid syndrome, Werner syndrome, vascular contributors to dementia, or cancer.

As used herein the adenosine A2a receptor agonists include related molecules, including variants in substituents, sidechains, and the like, that retain the capacity to reduce or ameliorate disease severity in patients having diseases and disabilities in an individual as a consequence of biological aging including but not limited to, specifically including but not limited to Alzheimer's Disease, Alzheimer's Disease Related Dementia (ADRD), Mixed Etiology Dementia (MED), Lewy Body dementia, fronto-temporal dementia, Progeroid syndromes, Hutchinson-Gilford progeroid syndrome, Werner syndrome, vascular contributors to dementia, or cancer. Such drugs also include structurally related drugs having improved specificity for any of the adenosine A2a receptors disclosed herein or known in the art to be associated with Alzheimer's Disease Related Dementia (ADRD), Mixed Etiology Dementia (MED), Lewy Body dementia, fronto-temporal dementia, Progeroid syndromes, Hutchinson-Gilford progeroid syndrome, Werner syndrome, vascular contributors to dementia, or cancer. Such alternative embodiments of adenosine A2a receptors associated with Alzheimer's Disease Related Dementia (ADRD) or Mixed Etiology Dementia (MED) include but are not limited to allelic variants or genetic variants found to be associated with associated with Alzheimer's Disease Related Dementia (ADRD), Mixed Etiology Dementia (MED), Lewy Body dementia, fronto-temporal dementia, Progeroid syndromes, Hutchinson-Gilford progeroid syndrome, Werner syndrome, vascular contributors to dementia, or cancer.

The invention also provides pharmaceutical compositions of drugs capable of reducing or ameliorating disease severity in an individual having diseases and disabilities in an individual as a consequence of biological aging including but not limited to Alzheimer's Disease Related Dementia (ADRD), Mixed Etiology Dementia (MED), Lewy Body dementia, fronto-temporal dementia, Progeroid syndromes, Hutchinson-Gilford progeroid syndrome, Werner syndrome, vascular contributors to dementia, or cancer. In specific embodiments the invention provides pharmaceutical compositions comprising therapeutically effective amounts of drugs capable of reducing or correcting dysfunctional expression of genes related to diseases and disabilities in an individual as a consequence of biological aging including but not limited to ADRD, MED, Lewy Body dementia, fronto-temporal dementia, Progeroid syndromes, Hutchinson-Gilford progeroid syndrome, Werner syndrome, vascular contributors to dementia, or cancer. As disclosed herein, such genes are identified from a neural organoid platform wherein administration of the drugs in vitro reduces or alters this dysfunctional gene expression, and accordingly ameliorates disease severity in an individual having diseases and disabilities in an individual as a consequence of biological aging including but not limited to ADRD, Mixed MED, Lewy Body dementia, fronto-temporal dementia, Progeroid syndromes, Hutchinson-Gilford progeroid syndrome, Werner syndrome, vascular contributors to dementia, or cancer, when the pharmaceutical composition is administered to such patients. In particular embodiments these genes are set forth in Tables 3-11 herein.

As used herein, the term “pharmaceutical composition” is intended to encompass and will be understood by those skilled in these arts to include agonists of adenosine A2a receptor, specifically adenosine A2a receptor having an amino acid sequence identified by GENBANK ACCESSION NO. NP_001265429.1 and encoded by ADORA2A (GENBANK ACCESSION NO. NM_001278497.2). Pharmaceutical compositions according to this invention include pharmacological salts, hydrates, or conjugates thereof. These compositions also include formulations, particularly formulations capable of traversing the blood-brain barrier and formulations that can be provided or oral administration for example in pill form. Alternative formulations, for example for injection, administration by inhalation, or rectal suppositories are also envisioned.

Specific embodiments of drugs comprising the active pharmaceutical ingredient (API) of the pharmaceutical compositions provided herein include methyl (1R,4r)-4-(3-(6-amino-9-((2R,3R,4S,5S)-5-(ethylcarbamoyl)-3,4-dihydroxytetrahydrofuran-2-yl)-9H-purin-2-yl)prop-2-yn-1-yl)cyclohexane-1-carboxylate (known in the art as apadenoson). In other specific embodiments, such drugs include 2-p-(2-Carboxyethyl) phenethylamino-5′-N-ethylcarboxamidoadenosine hydrochloride hydrate (known in the art as CGS-21680). In addition to such particular and specific embodiments this disclosure encompasses related drug molecules that are agonists of adenosine A2a receptors encoded by ADORA2A and allelic variants thereof, particularly such variants associated with aging-related diseases such as Alzheimer's Disease Related Dementia (ADRD), Mixed Etiology Dementia (MED), Lewy Body dementia, fronto-temporal dementia, Progeroid syndromes, Hutchinson-Gilford progeroid syndrome, Werner syndrome, vascular contributors to dementia, or cancer.

Additional embodiments of the methods and pharmaceutical compositions disclosed herein comprise combinations of adenosine A2a receptor agonists as disclosed herein as well as combinations of these drugs with other medicaments useful for reducing or ameliorating disease severity in an individual having age-related diseases such as Alzheimer's Disease Related Dementia (ADRD), Mixed Etiology Dementia (MED), Lewy Body dementia, fronto-temporal dementia, Progeroid syndromes, Hutchinson-Gilford progeroid syndrome, Werner syndrome, vascular contributors to dementia, or cancer. In specific embodiments such combinations include methyl (1R,4r)-4-(3-(6-amino-9-((2R,3R,4S,5S)-5-(ethylcarbamoyl)-3,4-dihydroxytetrahydrofuran-2-yl)-9H-purin-2-yl)prop-2-yn-1-yl)cyclohexane-1-carboxylate (known in the art as apadenoson), and/or 2-p-(2-Carboxyethyl) phenethylamino-5′-N-ethylcarboxamidoadenosine hydrochloride hydrate (known in the art as CGS-21680), in therapeutically useful combinations and dosages thereof, as well as combinations with alternative medicaments capable of reducing or ameliorating disease severity in an individual having diseases and disabilities as a consequence of biological aging including but not limited to Alzheimer's Disease Related Dementia (ADRD), Mixed Etiology Dementia (MED), Lewy Body dementia, fronto-temporal dementia, Progeroid syndromes, Hutchinson-Gilford progeroid syndrome, Werner syndrome, vascular contributors to dementia, or cancer.

In certain alternative embodiments, this invention also provides methods for reducing or ameliorating disease severity in an individual having diseases and disabilities as a consequence of biological aging, by treating individuals with pharmaceutical compositions of drugs capable of reducing or ameliorating disease severity in such an individual, specifically including but not limited to Alzheimer's Disease, Alzheimer's Disease Related Dementia (AD/ADRD), Mixed Etiology Dementia (MED), Lewy Body dementia, fronto-temporal dementia, Progeroid syndromes, Hutchinson-Gilford progeroid syndrome, Werner syndrome, vascular contributors to dementia, or cancer having a particular effect of such drugs on EZH2. EZH2 is a member of the Polycomb-group (PcG) family PRC2/EED-EZH2 complex, which methylates ‘Lys-9’ (H3K9me) and ‘Lys-27’ (H3K27me) of histone H3, leading to transcriptional repression and downstream targets SMARCA4 and KDM6A), KAT5 (a Lysine Acetyltransferase 5) and SETD7 (a Histone Lysine Methyltransferase). Epigenetic driving forces of aging include manipulation of the landscape of covalent chromatin modifications that result in changes in chromatin architecture, aberrant activation and silencing of specific gene sets and pathways. There are three major chromatin regulatory (or epigenetic) pathways, DNA methylation, histone acetylation and histone methylation. The regulation of these posttranslational modification of chromatin falls into three classes of chromatin regulatory proteins commonly termed writers, readers and erasers, which represent classes of enzymes that catalyze the placement of covalent modifications on DNA or histone substrates (‘write’), classes of proteins that utilizes specialized domains that recognize and bind to these modifications (‘read’) and enzymes that catalyze the removal of these modifications (‘erase’). Chromatin modifications are written, read and erased in a highly controlled and context-specific manner and represent mechanisms by which processes that require access to DNA such as transcription, DNA repair and replication are altered’ (Conery et al., Nat Chem Biol. 2022, 18; 124-133). By discerning their mechanism of action at the epigenetic and gene regulatory network (GRN) level, we have identified some novel epigenetic targets thus of utility to develop new chemical entities to treat aging. The effects of inhibiting the epigenetic target EZH2 with the drug Tazemetostat will be at a dose range of 10-100 nM (Ki and IC50 of 2.5 nM and 11 nM, exhibiting a 35-fold selectivity versus EZH). Tazemetostat, sold under the brand name Tazverik, is a FDA approved medication used for the treatment of adults and adolescents aged 16 years and older with metastatic (when cancer cells spread to other parts of the body) or locally advanced (when cancer has grown outside the organ it started in. Reference: Lue J K, Amengual J E (October 2018). “Emerging EZH2 Inhibitors and Their Application in Lymphoma”. Curr Hematol Malig Rep. 13 (5): 369-382.

As used herein, “combinations” can include formulations comprising one or more of the drugs specifically identified herein for reducing or ameliorating disease severity in an individual having diseases and disabilities in an individual as a consequence of biological aging, specifically including but not limited to Alzheimer's Disease, Alzheimer's Disease Related Dementia (ADRD), Mixed Etiology Dementia (MED), Lewy Body dementia, fronto-temporal dementia, Progeroid syndromes, Hutchinson-Gilford progeroid syndrome, Werner syndrome, vascular contributors to dementia, or cancer. In certain combinations the drugs are administered concomitantly and in other administration can be achieved over a specified time course. In some embodiments the combinations are provided in a single formulation whereas in others the drugs are separately formulated.

In certain embodiments the alternative medicament administered alone or in combination with adenosine A2a receptor agonists as disclosed herein are Fatty Acid Amide Hydrolase (FAAH) inhibitors, in specific embodiments wherein the FAAH inhibitor is PF-3845, CAS Name N-3-Pyridinyl-4-[[3-[[5-(trifluoromethyl)-2-pyridinyl]oxy]phenyl]methyl]-1-piperidinecarboxamide

Molecular Formula: C₂₄H₂₃F₃N₄O₂ Molecular Mass: 456.46, having the structure

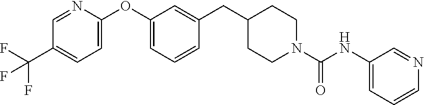 [SMILES: O=C(Nc1cccnc1)N1CCC(Cc2cccc(Oc3ccc(C(F)(F)F)cn3)c2)CC1]

SSR411298 (Clinical trials for SSR411298, EU Clinical Trials Register), or PF-622 (Ahn et al., 2007, Biochem. 46: 13019-13030). In certain alternative embodiments the alternative medicament administered in combination with adenosine A2a receptor agonists as disclosed herein are sphingosine-1-phosphate receptor 1 modulators, wherein specific sphingosine-1-phosphate receptor 1 modulators useful in these alternative medicaments are fingolimod, siponimed (Mayzant), ponesimed (ACT 12800), ceralifimod (ONO-4641), or Amiselmod (MT-1303).

As disclosed herein, genes have been identified from a neural organoid platform wherein administration of FAAH inhibitors in combination with adenosine A2a receptor agonists as disclosed herein in vitro reduces or alters this dysfunctional gene expression, and accordingly ameliorates disease severity in an individual having diseases and disabilities as a consequence of biological aging including but not limited to ADRD, Mixed MED, Lewy Body dementia, fronto-temporal dementia, Progeroid syndromes, Hutchinson-Gilford progeroid syndrome, Werner syndrome, vascular contributors to dementia, or cancer when the pharmaceutical composition is administered to such patients.

In further embodiments useful in the methods and provided in pharmaceutical compositions provided herein are adenosine A2b receptor antagonists, wherein the adenosine A2b receptor has an amino acid sequence is encoded by ADORA2B (GenBank Accession No. NM_000676.4). Specific embodiments of drugs comprising these adenosine A2b receptor antagonists include but are not limited to PBF-1129 and MRS-1706; see, Vazquez et al., 2008, “Local stimulation of the adenosine A2B receptors induces an increased release of IL-6 in mouse striatum: an in vivo microdialysis study,” J. Neurochem. 105: 904-9 and Ryzhov et I., 2008, “Effect of A2B adenosine receptor gene ablation on proinflammatory adenosine signaling in mast cells,” J. Immunol. 180: 7212-20.

As disclosed herein, genes have been identified from a neural organoid platform wherein administration of adenosine A2b receptor antagonists in vitro reduce or alter this dysfunctional gene expression, and accordingly ameliorates disease severity in an individual having diseases and disabilities as a consequence of biological aging including but not limited to ADRD, Mixed MED, Lewy Body dementia, fronto-temporal dementia, Progeroid syndromes, Hutchinson-Gilford progeroid syndrome, Werner syndrome, vascular contributors to dementia, or cancer when the pharmaceutical composition is administered to such patients. In particular embodiments these genes are set forth in Tables 3-11.

In further embodiments the skilled worker will understand that treatment with FAAH inhibitor PF-3845 will raise endogenous levels of endocannabinoid anandamide receptor agonist anandamide, wherein the endocannabinoid anandamide receptor has an amino acid sequence identified by GenBank Accession No. NP_001357474.1 and is encoded by CNR1 (GenBank Accession No. NM_016083.6). Consequently, PF-3845 treatment can increase endocannabinoid anandamide receptor activity despite dementia-associated downregulation and have effects on gene expression dysregulation affected by PF-3845 treatment. In particular embodiments these genes are set forth in Table 7 herein.

In further embodiments the skilled worker will understand that treatment with the alternative medicament administered alone or in combination with adenosine A2a receptor agonists as disclosed herein is rapamycin (and its analog Sirolimus; Blagosklonny, 2019, Aging, 11, 8048-8067) that inhibits mTOR. Mammalian target of rapamycin (mTOR) regulates cell proliferation, autophagy, and apoptosis by participating in multiple signaling pathways in the body, and many of which are associated with aging (Selvarani et al., 2021, Geroscience 43:1135-1158) The genes and cell signaling pathways related to the cell cycle, DNA repair cell death, mitochondria, immunity, nutrient signaling and the growth hormone Insulin Growth Factor-1 (IGF-1) mediated via the PI3K/AKT/mTOR (phosphoinositide 3-kinase/AKT (protein kinase B)/mammalian target for rapamycin) pathway have received extensive investigations as targets for anti-aging strategies (Mohammed et al., 2021, Front. Endocrinol. 12: 718942. Studies have shown that the mTOR signaling pathway is also associated with cancer, arthritis, insulin resistance, osteoporosis, and other diseases (Zhou et al., 2020, Cell Biosci 10:31). In Table 9, highlighted are some of the changes in gene expression correlated with aging from the PG-NNOP.

Rapamycin Formula: C51H79NO13; Molar mass: 914.187 has the structure:

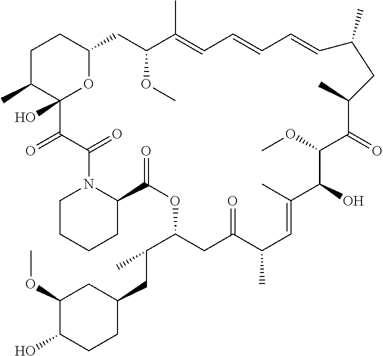 [SMILES: CO[C@H]1C[C@@H](C[C@H](C)[C@H]2CC(=O)[C@@H](C)/C=C(\C)[C@H](O)[C@H](OC)C(=O)[C@@H](C)C[C@@H](C)/C=C/C=C/C=C(\C)[C@H](OC)C[C@H]3CC[C@H](C)[C@](O)(O3)C(=O)C(=O)N3CCCC[C@@H]3C(=O)O2)CC[C@@H]1O]

In further embodiments the skilled worker will understand that treatment with the alternative medicament administered alone or in combination with adenosine A2a receptor agonists as disclosed herein is metformin that is considered of clinical utility for some age-related diseases (Glossman & Lutz, 2019, Metformin and Aging: A Review, Gerontology 65: 581-590) Metformin is a synthetic biguanide, orally effective and insulin sensitizing anti-diabetic drug. Metformin inhibits the inflammatory response through nuclear factor kB (NFkB) inhibition via pathways involving AMPK. Aging is a natural process, which plays a critical role in the pathogenesis of a variety of diseases, i.e., aging-related diseases, such as diabetes, osteoarthritis, Alzheimer disease, cardiovascular diseases, cancers, obesity and other metabolic abnormalities. The numerous beneficial health outcomes associated with the use of metformin to treat patients with type 2 diabetes (T2DM), together with data from pre-clinical studies in animals including the nematode, C. elegans, and mice have prompted investigations into whether metformin has therapeutic utility as an anti-aging drug that may also extend lifespan. Indeed, clinical trials, including the MILES (Metformin In Longevity Study) and TAME (Targeting Aging with Metformin), have been designed to assess the potential benefits of metformin as an anti-aging drug. Preliminary analysis of results from MILES indicate that metformin may induce anti-aging transcriptional changes; however, it remains controversial as to whether metformin is protective in those subjects free of disease (Mohammed et al., 2021, Id.). In Table 7, highlighted are some of the changes in gene expression correlated with aging from the PG-NNOP.

Metformin is a biguanide antihyperglycemic agent with Formula: C4H11N5; Molecular Mass: 129.167 and structure:

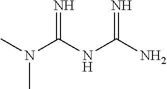

In Table 10 are identified changes in the expression of genes that are correlated with many biological and clinical symptoms or features. Exemplars include fibrosis that occurs in fibrotic heart and pulmonary disease (DOK5); hypertension (PTGIS); hyperinsulinemic hypoglycemia (SLC25A3); cancer (ATF3); sleep (SLC17A7) and dementia (SORL1).

These and other data findings, features, and advantages of the present disclosure will be more fully understood from the following detailed description taken together with the accompanying claims. It is noted that the scope of the claims is defined by the recitations therein and not by the specific discussion of features and advantages set forth in the present description.

### EXAMPLES

The Examples that follow are illustrative of specific embodiments of the invention, and the use thereof. It is set forth for explanatory purposes only and is not taken as limiting the invention. In particular, the example demonstrates the effectiveness of neural organoids in predicting future disease risk.

### Materials and Methods

The neural organoids described above were developed using the following materials and methods.

### Summary of Methods:

Neural Organoids derived from induced pluripotent stem cells derived from adult skin cells of AD, ADRD, MED, or Hutchinson Gilford progeria (PG) patients were grown in vitro for 4 weeks as previous described in U.S. Pat. No. 11,435,890, incorporated by reference in its entirety herein. Transcriptomic data from these neural organoids were obtained. Differences in expression of 20,814 genes expressed in the human genome were determined between these neural organoids and those from neural organoids from a normal individual human (an individual not having/diagnosed with AD, ADRD, MED, or Progeroid syndromes). Detailed data analysis using Gene Card and Pubmed data bases were performed. Genes that were expressed at greater than 1.4-fold were found to be highly significant because a vast majority were correlated with genes previously associated with a multitude of neurodevelopmental and neurodegenerative diseases as well as those found to be dysregulated in postmortem patient brains. These genes comprise a suite of biomarkers for Alzheimer's disease.

Cells used in these methods include human iPSCs, feeder-dependent (System Bioscience. WT SC600A-W) and CF-1 mouse embryonic fibroblast feeder cells, gamma-irradiated (Applied StemCell, Inc #ASF-1217)

Growth media, or DMEM media, used in the examples contained the supplements as provided in Table 1 (Growth Media and Supplements used in Examples).

### Table 1: Growth Media and Supplements Used in Examples

| Media/Supplement | Vendor/Catalog Number |
|---|---|
| DMEM non-essential amino acids | MEM-NEAA, Invitrogen #11140-050 |
| Phosphate Buffered Saline, sterile | Invitrogen #14040-091 |
| Phosphate Buffered Saline, | Invitrogen #14190-094 |
| Ca++ and Mg++ free | |
| Gentamicin Reagent Solution | Invitrogen #15750-060 |
| Antibiotic-Antimycotic | Invitrogen #15240-062 |
| 2-mercaptoethanol | EmbryoMAX, EMBMillipore#ES- |
| | 007-E |
| Basic fibroblast growth factor | FGF, PeproTech #051408-1 |
| Heparin | Sigma, #H3149-25KU |
| Insulin solution | Sigma #19278-5ml |
| Dimethyl sulfoxide | Millipore #D9170-5VL |
| ROCK Inhibitor Y27632 | Millipore#SCM075 |
| Gelatin solution, Type B | Sigma #GI 393-100ml |
| Matrigel Matrix NOT Growth | BD Bioscience #354234 |
| Factor Reduced Matrigel | |
| Accutase | Sigma #A6964 |
| Hydrogen Peroxide | Fisher #H325-500 |
| Ethanol | |
| Sterile H20 | |

One skilled in the art will recognize that additional formulations of media and supplements can be used to culture, induce and maintain pluripotent stem cells and neural organoids.

Experimental protocols required the use of multiple media compositions including MEF Media, IPSC Media, EB Media, Neural Induction Media, and Differentiation Medias 1, 2, and 3.

Mouse embryonic fibroblast (MEF) was used in cell culture experiments. MEF Media comprised DMEM media supplemented with 10% Feta Bovine Serum, 100 units/ml penicillin, 100 microgram/ml streptomycin, and 0.25 microgram/ml Fungizone.

Induction media for pluripotent stem cells (IPSC Media) comprised DMEM/F12 media supplemented with 20% Knockout Replacement Serum, 3% Fetal Bovine Serum with 2 mM Glutamax, IX Minimal Essential Medium Nonessential Amino Acids, and 20 nanogram/ml basic Fibroblast Growth Factor

Embryoid Body (EB) Media comprised Dulbecco's Modified Eagle's Medium (DMEM) (DMEM)/Ham's F-12 media, supplemented with 20% Knockout Replacement Serum, 3% Fetal Bovine Serum containing 2 mM Glutamax, IX Minimal Essential Medium containing Nonessential Amino Acids, 55microM beta-mercaptoethanol, and 4 ng/ml basic Fibroblast Growth Factor.

Neural Induction Media contained DMEM/F12 media supplemented with a 1:50 dilution N2 Supplement, a 1:50 dilution GlutaMax, a 1:50 dilution MEM-NEAA, and 10 microgram/ml Heparin’

Three differentiation media were used to produce and grow neural organoids. Differentiation Media 1 contained DMEM/F12 media and Neurobasal media in a 1:1 dilution. Each media is commercially available from Invitrogen. The base media was supplemented with a 1:200 dilution N2 supplement, a 1:100 dilution B27-vitamin A, 2.5 microgram/ml insulin, 55microM beta-mercaptoethanol kept under nitrogen mask and frozen at −20° C., 100 units/ml penicillin, 100 microgram/ml streptomycin, and 0.25 microgram/ml Fungizone.

Differentiation Media 2 contained DMEM/F12 media and Neurobasal media in a 1:1 dilution supplemented with a 1:200 dilution N2 supplement, a 1:100 dilution B27 containing vitamin A, 2.5 microgram/ml Insulin, 55umicroMolar beta-mercaptoethanol kept under nitrogen mask and frozen at −20° C., 100units/ml penicillin, 100 microgram/ml streptomycin, and 0.25 microgram/ml Fungizone.

Differentiation Media 3 consisted of DMEM/F12 media: Neurobasal media in a 1:1 dilution supplemented with 1:200 dilution N2 supplement, a 1:100 dilution B27 containing vitamin A), 2.5 microgram/ml insulin, 55microMolar beta-mercaptoethanol kept under nitrogen mask and frozen at −20° C., 100 units/ml penicillin, 100 microgram/ml streptomycin, 0.25 microgram/ml Fungizone, TSH, and Melatonin.

The equipment used in obtaining, culturing and inducing differentiation of pluripotent stem cells is provided in Table 2 (Equipment used in Experimental Procedures). One skilled in the art would recognize that the list is not at all exhaustive but merely exemplary.

### Table 2: Equipment Used in Experimental Procedures.

| | |
|---|---|
| StemPro EZPassage | Invitrogen#23181-010 |
| Tissue Culture Flasks, 115 cm² reclosable | TPP #TP90652 |
| Tissue Culture Flask, 150 cm² reclosable | TPP#TP90552 |
| Lipidure coat plate, 96 wells, U-bottom | LCU96 |
| Lipidure coat MULTI dish, 24 well | 510101619 |
| Parafilm | Sigma #P7793 |
| Sterile Filtration Units for 150 ml/250 ml | Sigma #TPP99150/ |
| solutions | TPP99250 |
| Benchtop Tissue Culture Centrifuge | ThermoFisher |
| C0₂ incubator, maintained at 37° C. and 5% | ThermoFisher |
| C0₂ | |
| Bench top rotary shaker | ThermoFisher |
| Light Microscope | Nikon |
| Confocal Microscope | Nikon |

### Example 1: Generation of Human Induced Pluripotent Stem Cell-Derived Neural Organoids

Human induced pluripotent stem cell-derived neural organoids were generated according to the following protocol, as set forth in U.S. Pat. No. 11,435,890, incorporated by reference in its entirety herein. Briefly, irradiated murine embryonic fibroblasts (MEF) were plated on a gelatin coated substrate in MEF media (Dulbecco's Modified Eagle Medium (DMEM) supplemented with 10% Fetal Bovine Serum, 100 units/ml penicillin, 100 microgram/ml streptomycin, and 0.25 microgram/ml Fungizone) at a density of 2×10⁵ cells per well. The seeded plate was incubated at 37° C. overnight.

After incubation, the MEFs were washed with pre-warmed sterile phosphate buffered saline (PBS). The MEF media was replaced with 1 mL per well of induced pluripotent stem cell (iPSC) media containing Rho-associated protein kinase (ROCK) inhibitor. A culture plate with iPSCs was incubated at 37° C. The iPSCs were fed every other day with fresh iPSC media containing ROCK inhibitor. The iPSC colonies were lifted, divided, and transferred to the culture wells containing the MEF cultures so that the iPSC and MEF cells were present therein at a 1:1 ratio. Embryoid bodies (EB) were then prepared. Briefly, a 100 mm culture dish was coated with 0.1% gelatin and the dish placed in a 37° C. incubator for 20 minutes, after which the gelatin-coated dish was allowed to air dry in a biological safety cabinet. The wells containing iPSCs and MEFs were washed with pre-warmed PBS lacking Ca²⁺/Mg²⁺. A pre-warmed cell detachment solution of proteolytic and collagenolytic enzymes (1 mL/well) was added to the iPSC/MEF cells. The culture dishes were incubated at 37° C. for 20 minutes until cells detached. Following detachment, pre-warmed iPSC media was added to each well and gentle agitation used to break up visible colonies. Cells and media were collected and additional pre-warmed media added, bringing the total volume to 15 mL. Cells were placed on a gelatin-coated culture plate at 37° C. and incubated for 60 minutes, thereby allowing MEFs to adhere to the coated surface. The iPSCs present in the cell suspension were then counted.

The suspension was then centrifuged at 300×g for 5 minutes at room temperature, the supernatant discarded, and cells re-suspended in EB media supplemented with ROCK inhibitor (50 uM final concentration) and 4 ng/ml basic Fibroblast Growth Factor to a volume of 9,000 cells/150 μL. EB media is a mixture of DMEM/Ham's F-12 media supplemented with 20% Knockout Replacement Serum, 3% Fetal Bovine Serum (2 mM Glutamax), 1× Minimal Essential Medium Nonessential Amino Acids, and 55 μM beta-mercaptoethanol. The suspended cells were plated (150 μL) in a LIPIDURE® low-attachment U-bottom 96-well plate and incubated at 37° C.

The plated cells were fed every other day during formation of the embryoid bodies by gently replacing three fourths of the embryoid body media without disturbing the embryoid bodies forming at the bottom of the well. Special care was taken in handling the embryoid bodies so as not to perturb the interactions among the iPSC cells within the EB through shear stress during pipetting. For the first four days of culture, the EB media was supplemented with 50 uM ROCK inhibitor and 4 ng/ml beta-fibroblast growth factor (bFGF). During the remaining two to three days the embryoid bodies were cultured, no ROCK inhibitor or bFGF was added.

On the sixth or seventh day of culture, the embryoid bodies were removed from the LIPIDURE® 96 well plate and transferred to two 24-well plates containing 500 μL/well Neural Induction media, DMEM/F12 media supplemented with a 1:50 dilution N2 Supplement, a 1:50 dilution GlutaMax, a 1:50 dilution MEM-Non-Essential Amino Acids (NEAA), and 10 μg/ml Heparin. Two embryoid bodies were plated in each well and incubated at 37° C. The media was changed after two days of incubation. Embryoid bodies with a “halo” around their perimeter indicate neuroectodermal differentiation. Only embryoid bodies having a “halo” were selected for embedding in Matrigel, remaining embryoid bodies were discarded.

Plastic paraffin film (PARAFILM) rectangles (having dimensions of 5 cm×7 cm) were sterilized with 3% hydrogen peroxide to create a series of dimples in the rectangles. This dimpling was achieved, in one method, by centering the rectangles onto an empty sterile 200 μL tip box press and pressing the rectangles gently to dimple it with the impression of the holes in the box. The boxes were sprayed with ethanol and left to dry in the biological safety cabinet.

Frozen Matrigel matrix aliquots (500 μL) were thawed on ice until equilibrated at 4° C. A single embryoid body was transferred to each dimple of the film. A single 7 cm×5 cm rectangle holds approximately twenty (20) embryoid bodies. Twenty microliter (20 μL) aliquots of Matrigel were transferred onto the embryoid bodies after removing extra media from the embryoid body with a pipette. The Matrigel was incubated at 37° C. for 30 min until the Matrigel polymerized. The 20 μL droplet of viscous Matrigel was found to form an optimal three-dimensional environment that supported the proper growth of the neural organoid from embryoid bodies by sequestering the gradients of morphogens and growth factors secreted by cells within the embryoid bodies during early developmental process. However, the Matrigel environment permitted exchange of essential nutrients and gases. Gentle oscillation by hand twice a day for a few minutes within a tissue culture incubator (37° C./5% CO₂) further allowed optimal exchange of gases and nutrients to the embedded embryoid bodies.

Differentiation Media 1, a one-to-one mixture of DMEM/F12 and Neurobasal media supplemented with a 1:200 dilution N2 supplement, a 1:100 dilution B27-vitamin A, 2.5 μg/mL insulin, 55 μM beta-mercaptoethanol kept under nitrogen mask and frozen at −20° C., 100 units/mL penicillin, 100 μg/mL streptomycin, and 0.25 μg/mL Fungizone, was added to a 100 mm tissue culture dish. The film containing the embryoid bodies in Matrigel was inverted onto the 100 mm dish with differentiation media 1 and incubated at 37° C. for 16 hours. After incubation, the embryoid body/Matrigel droplets were transferred from the film to the culture dishes containing media. Static culture at 37° C. was continued for 4 days until stable neural organoids formed.

Organoids were gently transferred to culture flasks containing differentiation media 2, a one-to-one mixture of DMEM/F12 and Neurobasal media supplemented with a 1:200 dilution N2 supplement, a 1:100 dilution B27+vitamin A, 2.5 μg/mL insulin, 55microM beta-mercaptoethanol kept under nitrogen mask and frozen at −20° C., 100 units/mL penicillin, 100 μg/mL streptomycin, and 0.25 μg/mL Fungizone. The flasks were placed on an orbital shaker rotating at 40 rpm within the 37° C./5% CO₂ incubator.

The media was changed in the flasks every 3-4 days to provide sufficient time for morphogen and growth factor gradients to act on targets within the recipient cells forming relevant structures of the brains. Great care was taken when changing media so as to avoid unnecessary perturbations to the morphogen/secreted growth factor gradients developed in the outer most periphery of the organoids as the structures grew into larger organoids.

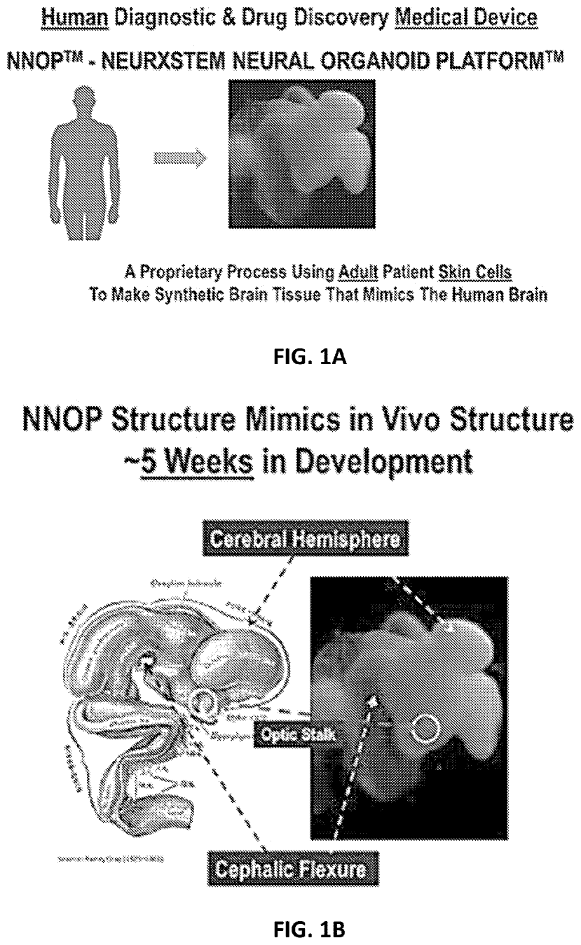

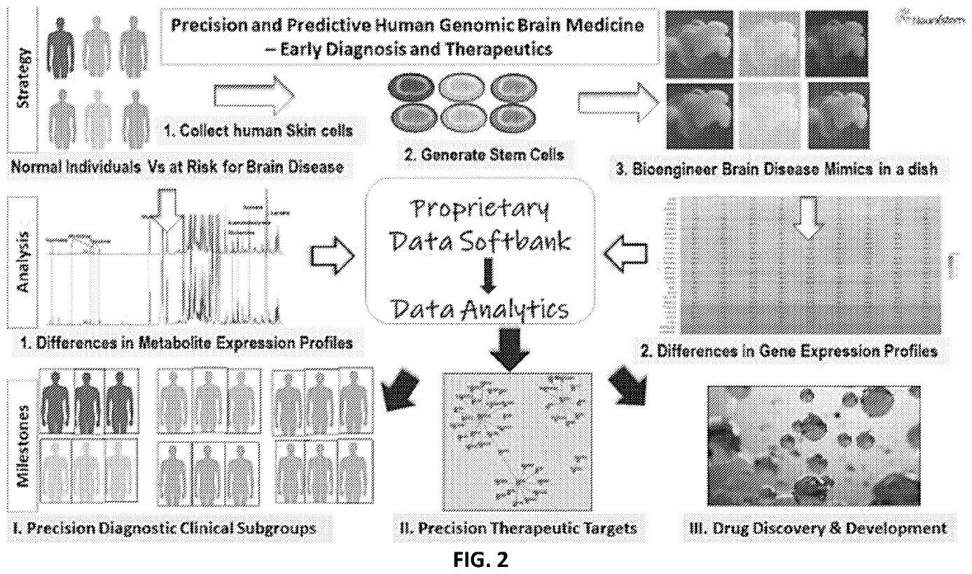

FIG. 1 illustrates neural organoid development in vitro. Based on transcriptomic analysis, iPSC cells form a body of cells after 3D culture, which become neural progenitor cells (NPC) after neural differentiation media treatment. Neurons were observed in the cell culture after about one week. After about four (4) weeks or before, neurons of multiple lineage appeared. At about twelve (12) weeks or before, the organoid developed to a stage having different types of cells, including microglia, oligodendrocyte, astrocyte, neural precursor, neurons, and interneurons.

### Example 2: Human Induced Pluripotent Stem Cell-Derived Neural Organoids Expressed Characteristics of Human Brain Development and the Reproducibility, Replicability, and Robustness of the NNOP Model

After approximately 12 weeks of in vitro culture, transcriptomic and immunohistochemical analysis indicated that organoids were generated according to the methods delineated in Example 1. Specifically, the organoids contained cells expressing markers characteristic of neurons, astrocytes, oligodendrocytes, microglia, and vasculature (FIGS. 1A and 1B) and all major brain structures of neuroectodermal derivation. Morphologically identified by bright field imaging, the organoids included readily identifiable neural structures including cerebral cortex, cephalic flexure, and optic stalk (compare, Grey's Anatomy Textbook). The gene expression pattern in the neural organoid was >98% concordant with those of the adult human brain reference (Clontech, #636530). The organoids also expressed genes in a developmentally organized manner described previously (e.g. for the midbrain mesencephalic dopaminergic neurons; Blaese et al., Genetic control of midbrain dopaminergic neuron development. Rev Dev Biol. 4(2): 113-34, 2015). The structures also stained positive for multiple neural specific markers (dendrites, axons, nuclei), cortical neurons (Doublecortin), midbrain dopamine neurons (Tyrosine Hydroxylase), and astrocytes (GFAP) as shown by immunohistology).

All human neural organoids were derived from iPSCs of fibroblast origin (from Coriell Biorepository, NJ). The development of a variety of brain structures was characterized in the organoids. Biomarkers specific for particular regions of human brain were detected as set forth in U.S. Pat. No. 11,435,890, incorporated by reference in its entirety herein.

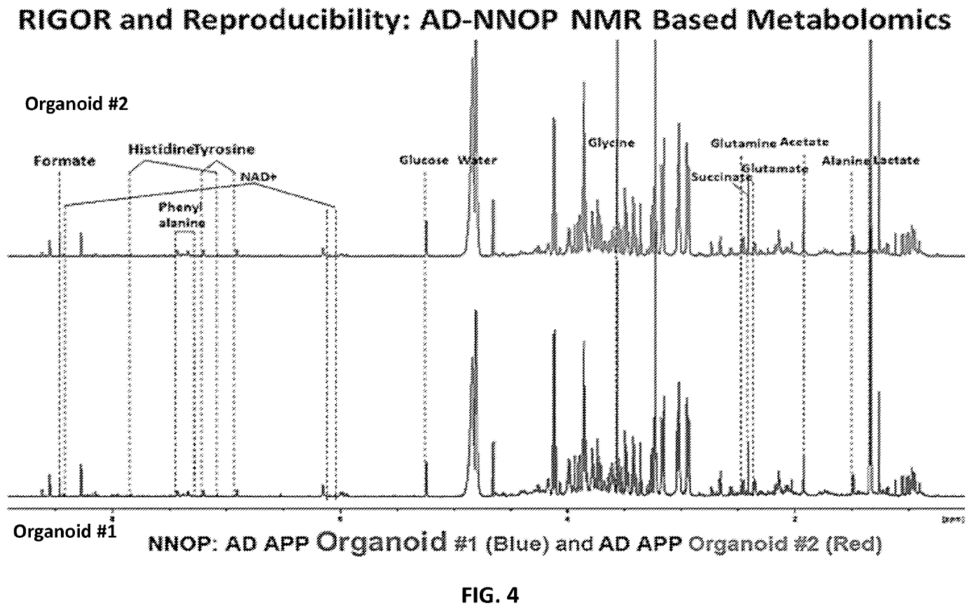

The results of transcriptomics experiments are shown in FIG. 4 and were performed as described herein and in U.S. Pat. No. 11,435,890, incorporated by reference in its entirety. Briefly, neural organoids were cultured in media after neural differentiation was initiated with addition of retinoic acid as per the published protocol (International patent application publication No. WO2017123791A1). Cultures were replenished with fresh media every week. Organoids were harvested after D0, D3, Week 1, Week 4 and Week 12 in culture. The two random independent neural organoids from the same ADRD/MED-APP patient showed consistently the same metabolic profile, which demonstrates the reproducibility, replicability, and feasibility of tracking changes in metabolites of interest in age-related changes including AD/ADRD in response to any therapeutic drug.

FIG. 3 showed transcriptomic data of about 15,000 genes of NNOP obtained from familial (APP) and sporadic (SPOR) patients in controlled clinical studies. Each dot represented RNA expression level of a gene measured in two randomly selected NNOP out of hundreds of NNOP. The variance was as low as <0.95 showing that there was little variability between independent organoid replicates, which demonstrated the reproducibility, replicability, and robustness of the NNOP model. Thus, NNOP models are a reliable model to study AD specifically and aging-related diseases in general, and comparative data analysis from NNOP platform between normal and dementia patient-derived samples permits identification of therapeutic targets and individualization of treatment decisions.

FIG. 5 shows AD-NOP transcriptomic data illustrating significant overlap with transcriptomic data from accelerated aging Progeroid syndromes patient sample-derived PG-NNOP. Expression of differentially expressed genes in PG-NNOP and four AD-NOP neural organoids are compared to Normal-NOP in culture for 12 weeks. RNA extraction and gene expression analysis performed by Ampliseq™ (Thermofisher) as set forth herein. The four AD-NOP models (AD-NOP-APP; AD-NOP-PSEN2; AD-NOP-SPOR; and AD-NOP-ApoE) were derived from AD patient donors and the iPSCs obtained from the Coreill Biorepository (NJ, USA). Clinical validation of NNOP for AD/ADRD is apparent from significant corroboration of AD-APP-NOP transcriptomic data with clinical postmortem AD brain transcriptomic data analyses (Annese et al. Sci Rep. 2018; 8(1):4282) of a match with a statistical p-value of 1e⁻¹⁰³ of a match by chance alone.

Similar to FIG. 3, FIG. 6 shows normal-NNOP & PG-NNOP having highly replicable, reliable, and robust transcriptomic data. Independent NNOP sample transcriptomic at week 12 in culture. Plots represent high data reproducibility for ~13,000 genes expressed with a variance of <0.95 in independent replicates. These data are shown in Tables 5-10 herein.

### Example 3: NNOP Models Identified Common Biological Markers and Therapeutic Targets in Aging Related Diseases

Transcriptomic analysis of differentially expressed genes (DEG) in PG-NNOP and AD-NNOP (APP, PSEN2, SPOR, and ApoE models) compared to normal NNOP revealed many common biological markers of both diseases (Table 5A and Table 5B). Furthermore, the transcriptomic data collected from AD-NNOP and PG-NNOP models could also provide information regarding potential therapeutic targets. For example, based on STRING network analysis, some DEGs in AD-NNOP-APP and PG-NNOP (compared to Normal-NNOP) were identified to interact with each other. Namely, changes in expression of representative genes in the ADORA2A STRING Network (Genecards) is shown in the following Table 3 and Table 4 for APP-NNOP and PG-NNOP respectively. Additionally, based on the data collected from AD-NNOP models compared to Normal-NNOP and mathematical modeling, five key biomarkers identified in Table 11 were predicted to be effective therapeutic targets to treat AD. These were: CNR1 encoding endocannabinoid Anandamide Receptor; ADORA2A, ADORA1, ADORA2B all encoding adenosine receptors; and S1PR1 encoding sphingosine-1-phosphate-receptor. Thus, drugs that modulate these targets could correct the gene dysregulation in AD-NNOP models and clinical AD. The results of Table 3, 4, 5A, Table 5B, Table 11, suggested that NNOP model could be used to identify prominent biological markers in aging related diseases and potential targets for drug treatment.

| Gene_ID | base Mean | log2 Fold Change | p-value |
|---|---|---|---|
| ADORA1 | 150.83 | 1.94 | 0.0108 |
| ADORA2A | 30.95 | 2.17 | 0.0311 |
| CALM3 | 9148.53 | 0.82 | 0.0010 |
| SYNGR1 | 404.15 | 1.85 | 0.0001 |

Table 3. Neural organoids were cultured in media after neural differentiation was initiated with addition of retinoic acid as per the published protocol (USPTO patent WO2017123791A1). The cultures were replenished with fresh media every week. Organoids were harvested after 12 weeks in culture and subject to transcriptomic analysis. Results (log 2 fold change) show are normal-NNOP compared to AD-NNOP-APP.

| Gene_ID | base Mean | log2 Fold Change | p-value |
|---|---|---|---|
| ADORA1 | 132.99 | 2.92 | 4.401E−07 |
| CALM3 | 10079.12 | 0.72 | 0.0004116 |
| GRM5 | 27.02 | 5.70 | 6.73E−05 |
| NECAB2 | 109.92 | −2.11 | 0.000143 |
| SYNGR1 | 413.51 | 1.62 | 9.678E−06 |

Table 4. Neural organoids were cultured in media after neural differentiation was initiated with addition of retinoic acid as per the published protocol (USPTO patent WO2017123791A1). The cultures were replenished with fresh media every week. Organoids were harvested after 12 weeks in culture and subject to transcriptomic analysis. Results (log 2 fold change) show are normal-NNOP compared to PG-NNOP.

### Example 4: Testing Adenosine A2a Receptor Agonists and Endocannabinoid Anandamide Receptor Agonists for Efficacy in Correcting Dysfunctional Expression of Biomarkers Involved in Aging in Human NNOP Model

Based on the results in Table 3, 4, and 11, targeting the ADORA pathway by adenosine agonists (A_{2A}) were tested for effects on gene expression dysregulation in NNOP platform cells as set forth herein.

2-p-(2-Carboxyethyl) phenethylamino-5′-N-ethylcarboxamidoadenosine hydrochloride hydrate (CGS-21680) is a specific adenosine A_{2A} subtype receptor agonist (CAS Number:124182-57-6; Molecular Weight:535.98 (anhydrous basis), available from Sigma Chemical Co.). It is usually presented as an organic hydrochloride salt with a molecular weight of 536.0 g/M, is soluble up to 3.4 mg/mL in DMSO and 20 mg/mL in 45% (w/v) aq 2-hydroxypropyl-β-cyclodextrin.

CGS-21680 was tested at a concentration of 1 micromolar to assess the number of disease modifying genes that are differentially expressed to ‘normalize’ their expression by epigenetic mechanism affecting gene regulatory networks in ADRD/MED. The results of these experiments are shown in Table 6. Table 6 shows gene expression changes in NNOP cultures developed from iPSCs from adult skin cells from a patient having Alzheimer's disease with ApoE variant (ApoE4). Progeroid syndromes genetic and ApoE4 allele risk carriers are both considered models of human aging. The association between decline in physical function and age-related conditions, such as reduced cognitive performance and vascular disease, may be explained by genetic influence on shared biological pathways of importance for aging. The apolipoprotein E (APOE) gene is well-known for its association with Alzheimer's disease, reduced cognitive performance and grip strength, which is a key component of sarcopenia and frailty, where grip strength is an estimate of overall muscle strength (Skoog et al., 2016, Front. Aging Neurosci. 8:225). Addition of CGS-21680 significantly altered gene expression that were dysregulated in both AD-ApoE-NNOP and PG-NNOP. Furthermore, the changes were shifted toward normalcy when compared to the gene expression in PG-NNOP without the drug treatment, suggesting that CGS-21680 is effective in correcting the dysregulated gene expression in PG-NNOP and aging diseases in general.

Metformin and rapamycin were tested in PG-NNOP directly and they also shifted the gene expression towards normalcy (Table 8 and Table 9 respectively).

Based on the results of Table 11, endocannabinoid anandamide receptor was shown to be significantly altered in AD. Thus, given the overlap in transcriptomic profile of AD and PG, experiments were set out to test the effect of targeting this receptor on PG-NNOP. FAAH inhibitor PF-3845, which is known to raise endogenous levels of endocannabinoid anandamide receptor agonist anandamide and increase endocannabinoid anandamide receptor activity, was tested for effects on gene expression dysregulation in PG-NNOP platform cells as set forth herein (Table 7). The results in Table 7 showed that PF-3845 was effective in correcting the gene expression that was altered in PG-NNOP. As the log 2 fold numbers reveal, the direction of the change in gene expression was completely reversed in some biomarkers by PF-3845, or the change was reduced by PF-3845. Thus, PF-3845 was capable of reverting the biomarkers of PG to normalcy.

These results show that the same genes that were ‘normalized” by A_{2A} receptor agonists (CGS-21680, metformin, and rapamycin) and FAAH inhibitor (PF-3845), were dysregulated in both Alzheimer and PG disease (Table 5A and Table 5B). This suggests that these drugs can be effective in correcting expression of genes related in aging-related diseases in general. Also set forth in the tables are references showing associations between each of the curated genes identified as having gene expression dysregulation in NNOP generated from iPSC prepared from adult skin cells of AD and WRN patients and their relevance to aging phenotypes. Of note that the curated genes in Tables 3-13 were not the only ones but they were some of thousands of genes that were found to be significantly altered in aging-related diseases (PG and AD) and respond to the selected drug treatments.

Another adenosine A_{2A} receptor agonist, apadenoson (ATL-146e), chemical name methyl (1R,4r)-4-(3-(6-amino-9-((2R,3R,4S,5S)-5-(ethylcarbamoyl)-3,4-dihydroxytetrahydrofuran-2-yl)-9H-purin-2-yl)prop-2-yn-1-yl)cyclohexane-1-carboxylate; CAS Number: 250386-15-3 Molecular Weight: 486.529; Soluble in DMSO. MedKoo Biosciences, Inc., is a selective adenosine A_{2A} receptor agonist and a potent inhibitor of inflammation. Apadenoson is tested using a range of concentrations (dose range: 100 nM-1microM; EC50 of 110 nM); to enhance the effects of adenosine apadenoson certain experiments are performed in the presence of Rolipram (1-10microM; IC₅₀=800 nM). The experiments will be carried out and results statistically analyzed as described above.

### Example 5: Gene Therapy to Modify Expression of Adenosine Receptor Network Components and/or Other Aging-Related Genes

Because pharmacological modulation of the adenosine receptor and endocannabinoid anandamide receptor activities resulted in changes in gene expression of aging biological markers as shown above, gene therapy using genetic engineering tools such as anti-sense oligonucleotide, microRNA, or CRISPR to modify expression of ADORA2A or CNR1 should bring about the same effect. Furthermore, using these tools to modulate expression of genes listed in Table 3-11 should also correct or reduce dysfunctional expression of genes related to aging-related diseases or aging-related comorbidities.

### Example 6: Aging Biomarkers Expressed Outside the Brain as Potential Diagnostic Tools

Table 10 shows differentially expressed genes that were identified in fibroblast samples obtained from AD patients compared to normal patients. These genes overlapped with gene expression that was altered in PG-NNOP and were known to associate with some clinical symptoms of aging. These results suggest that aging biomarkers are potentially present throughout different organs and tissues in patients with aging-related diseases, and not restricted to just the aging organs and tissues. This means that a sample from easily accessed tissues such as skin and blood can be collected and screened for aging biomarkers. The PG-NNOP can be further generated from the samples (such as fibroblast) and screened for additional differentially expressed genes that are related to aging. These transcriptomic data can then assist diagnosis and determination of aging risks, and/or selection of appropriate treatment of the aging-related diseases or symptoms.

### Example 7: Testing Adenosine A2a Receptor Agonists for Efficacy in Correcting Dysfunctional Expression of Biomarkers Involved in Nutritional Uptake and Susceptibility to Environmental Toxins in Human NNOP Model

Nutrition, particularly maternal and postnatal nutrition during embryonic development, and environmental exposure play a role in healthy aging (Leitao et al., 2022, Nutrients 14(3): 554). Some genes involved in nutritional uptakes such as SELENBP1 and RARB were found to be differentially expressed in PG-NNOP and AD-ApoE-NNOP compared to Normal-NNOP (Table 12). SELENBP1, is a selenium-binding protein family. Selenium is an essential nutrient that exhibits potent anticarcinogenic properties, and deficiency of selenium may cause certain neurologic diseases. The effects of selenium in preventing cancer and neurologic diseases may be mediated by selenium-binding proteins, and decreased expression of this gene may be associated with several types of cancer. Meanwhile, RARB (Retinoic Acid Receptor Beta) binds retinoic acid, the biologically active form of vitamin A which mediates cellular signaling in embryonic morphogenesis, cell growth and differentiation. Treatment with CGS-21680 reduced the changes in expression of both genes in AD-ApoE-NNOP in the direction towards normalcy (Table 13). The results demonstrated that certain genes involved in nutritional uptake can be dysregulated before birth and potentially affect fetal development in utero since the neural organoid reflects the transcriptomic profile during the embryonic stage. Adjusting maternal nutritional care and pharmacological intervention to correct the gene expression can mitigate at least some of these dysfunctions and their effects on pre-mature aging later in life.

In addition, some genes involved in detoxification and susceptibility to environmental toxins are important in determining risk factors that affect longevity. For examples, SLC7A5, a high-affinity transporter that mediates uptake of large neutral amino acids. The complex formed by SLC3A2 and SLC7A5/LAT1 plays a role in hepatitis C virus/HCV propagation by facilitating viral entry into host cell and increasing L-leucine uptake-mediated mTORC1 signaling activation, thereby contributing to HCV-mediated pathogenesis. MT2A, a metallothionine, is involved in detoxification of cadmium found in particulates. Both genes were found to be dysregulated in PG-NNOP and AD-ApoE (Table 12), and the dysregulation was corrected by CGS-21680 treatment (Table 13). The data suggest that the neural organoid model can be used to identify genes involved in a patient's selective vulnerability to infectious agents or environmental toxins.

One of skill in the art will recognize that sequence data for the genes listed above can be obtained in publicly available gene databases such as GeneCards, GenBank, Malcard, Uniport and PathCard databases. The skilled worker will recognize these markers as set forth exemplarily herein to be human-specific marker proteins as identified, inter alia, in genetic information repositories such as GenBank; Accession Number. One having skill in the art will recognize that variants derive from the full length gene sequence.

| | | | | W12 AD AD-NNOP-ApoE, | | |
|---|---|---|---|---|---|---|
| | W12 PG-NNOP | | | -PSEN2, -APP, -SPOR | | |
| | | Log2 | | | Log2 | |
| | Base | Fold | | Base | Fold | |
| Genes | Mean | Change | P-Value | Mean | Change | P-Value |

DNA Stability

| | | | | | | |
|---|---|---|---|---|---|---|
| POGZ | 2220.58 | 0.49 | 0.002381952 | 2036.45 | 0.71 | 0.00074 |
| POLE3 | 1110.51 | −1.18 | 7.16121E−05 | 535.11 | 0.60 | 0.04622 |
| XPA | 369.32 | −1.48 | 9.77593E−07 | 275.44 | −0.92 | 0.00176 |

Telomer Attrition

| | | | | | | |
|---|---|---|---|---|---|---|
| TERF2IP | 4430.76 | −0.35 | 0.024840787 | 216.92 | −1.50 | 0.01257 |
| USP36 | 1244.66 | −1.51 | 1.4806E−10 | 849.56 | −0.82 | 0.00198 |

Epigenetic Alterations

| | | | | | | |
|---|---|---|---|---|---|---|
| EZH2 | 907.74 | 0.43 | 0.033867159 | 528.95 | 2.30 | 2.8E−10 |
| HDAC2 | 3526.21 | 0.68 | 0.000103534 | 2982.00 | 1.14 | 4.7E−06 |
| KAT6B | 293.84 | 1.27 | 0.000178679 | 318.64 | 0.71 | 0.03427 |
| KDM3A | 763.34 | −0.68 | 0.045074035 | 837.55 | −0.94 | 0.01197 |

Loss of Proteostasis

| | | | | | | |
|---|---|---|---|---|---|---|
| DNAJB2 | 565.74 | −0.61 | 0.04371453 | 335.38 | 0.80 | 0.0219 |
| DNAJC1 | 1356.07 | −1.16 | 1.17545E−09 | 514.00 | −0.60 | 0.00788 |
| DNAJC6 | 1012.68 | 1.55 | 1.3776E−12 | 1104.16 | 0.87 | 0.0261 |
| USP31 | 654.86 | 1.15 | 0.00013359 | 626.70 | 1.23 | 1.7E−05 |
| VCP | 5661.55 | −0.55 | 0.007664303 | 4507.35 | −0.53 | 0.01396 |

Deregulated Nutrient Sensing

| | | | | | | |
|---|---|---|---|---|---|---|
| CERS6 | 2276.65 | 1.03 | 8.96819E−09 | 2493.10 | 0.57 | 0.00093 |
| MCHR1 | 29.32 | 2.43 | 0.022887108 | 29.67 | 2.04 | 0.04472 |
| SESN2 | 746.95 | −0.41 | 0.040385672 | 678.99 | −0.77 | 0.03859 |

Cellular Senescence

| | | | | | | |
|---|---|---|---|---|---|---|
| CDK1 | 542.98 | 0.84 | 0.009770557 | 561.68 | 0.61 | 0.0256 |
| CDKN1A | 4290.40 | −2.56 | 5.95694E−25 | 2538.49 | −1.71 | 1.7E−07 |
| LMNB2 | 627.59 | 1.28 | 1.22146E−08 | 691.60 | 0.75 | 0.00025 |

Stem Cell Exhaustion

| | | | | | | |
|---|---|---|---|---|---|---|
| SLC7A1 | 740.88 | 0.8 | 0.027493639 | 719.78 | 0.82 | 0.1353 |

Mitochondrial Dysfunction

| | | | | | | |
|---|---|---|---|---|---|---|
| TOMM40 | 670.56 | −0.95 | 0.007597837 | 712.70 | −1.13 | 0.00184 |

Altered Intercellular Communication

| | | | | | | |
|---|---|---|---|---|---|---|
| ALDOC | 545.25 | −1.28 | 0.000166961 | 185.53 | 2.17 | 3.9E−05 |
| MT2A | 1533.21 | −3.20 | 7.18952E−13 | 188.44 | 1.81 | 0.00323 |
| SLC2A3 | 1603.73 | −0.91 | 0.009171 | 1902.46 | −1.32 | 0.00481 |
| SLC35F1 | 430.35 | 1.15 | 0.000370603 | 282.28 | 6.76 | 4.7E−20 |
| SLC38A1 | 323.56 | 1.65 | 1.5103E−08 | 319.12 | 1.43 | 0.00226 |

Disabled Macrophagy

| | | | | | | |
|---|---|---|---|---|---|---|
| SQSTM1 | 1874.47 | −0.57 | 0.001952166 | 11219.55 | −3.87 | 5.8E−25 |

Chronic Inflammation

| | | | | | | |
|---|---|---|---|---|---|---|
| NLRC5 | 74.79 | 2.64 | 0.005977849 | 71.92 | 2.81 | 0.00014 |

Dysbiosis

| | | | | | | |
|---|---|---|---|---|---|---|
| NAPEPLD | 557.99 | 0.79 | 0.036067167 | 558.93 | 0.68 | 0.02523 |
| TLR4 | 19.72 | 4.18 | 0.002324116 | 22.04 | 2.27 | 0.03162 |

Immunity

| | | | | | | |
|---|---|---|---|---|---|---|
| ID2 | 1712.85 | 2.37 | 6.13947E−55 | 2497.66 | 0.35 | 0.0393 |
| IDI1 | 2453.50 | −1.61 | 3.53894E−10 | 1633.28 | −0.82 | 0.00704 |

Progeroid syndromes (Accelerated Aging)

| | | | | | | |
|---|---|---|---|---|---|---|
| WRN | 381.53 | −0.85 | 0.010555836 | 387.20 | −0.99 | 0.00235 |
| DHX9 | 888.04 | 1.22 | 7.03735E−09 | 1027.00 | 0.52 | 0.00278 |

### Table 5A. Shared Novel Progeroid Syndrome and AD/ADRD Aging Biomarkers

Expression of differentially expressed genes in PG-NNOP and four AD-NNOP neural organoids compared to Normal-NNOP in culture for 12 weeks. RNA extraction and gene expression analysis performed by Ampliseq™ (Thermofisher). The four AD-NNOP models (AD-NNOP-APP; AD-NNOP-PSEN2; AD-NNOP-SPOR; and AD-NNOP-ApoE) were derived from AD patient donors and the iPSCs obtained from the Coreill Biorepository (NJ, USA). Log 2 fold change with a positive value indicates a decrease in expression of an AD-NNOP or PG-NNOP gene when compared to Normal-NNOP and with a negative number an increase in gene expression.

| | W12 PG-NNOP | | | W12 AD AD-NNOP-ApoE | | |
|---|---|---|---|---|---|---|
| | | Log2 | | | Log2 | |
| | Base | Fold | | Base | Fold | |
| Genes | Mean | Change | P-Value | Mean | Change | P-Value |

Osteoarthritis

| | | | | | | |
|---|---|---|---|---|---|---|
| SPP1 | 150.89 | 2.59 | 1.48E−07 | 634.56 | 6.04 | 9.67E−54 |

Migraine

| | | | | | | |
|---|---|---|---|---|---|---|
| ZEB2 | 1839.41 | 2.08 | 1.44E−11 | 839.30 | 2.08 | 7.77E−05 |
| PLCE1 | 473.49 | 1.50 | 4.03E−07 | 217.06 | 0.97 | 3.80E−02 |

Atherosclerosis

| | | | | | | |
|---|---|---|---|---|---|---|
| ANGPT1 | 105.40 | 1.98 | 4.16E−03 | 76.26 | 2.38 | 1.56E−04 |

Ceramides and Aging

| | | | | | | |
|---|---|---|---|---|---|---|
| SPTSSA | 498.17 | −0.83 | 1.06E−02 | 907.52 | 0.49 | 4.34E−02 |
| PLEKHA8 | 397.31 | 0.75 | 3.37E−02 | 918.95 | −0.73 | 4.33E−02 |

Neuropathic Pain

| | | | | | | |
|---|---|---|---|---|---|---|
| AK2 | 908.24 | −0.93 | 3.56E−05 | 1327.85 | 1.40 | 2.80E−06 |

Inherited Cancer Syndrome

| | | | | | | |
|---|---|---|---|---|---|---|
| DHFR | 478.36 | 1.15 | 7.98E−03 | 1266.49 | 2.04 | 1.44E−11 |

Heart Disease

| | | | | | | |
|---|---|---|---|---|---|---|
| BHLHE40 | 1030.35 | −1.51 | 8.63E−03 | 690.49 | 3.19 | 5.36E−12 |
| ABCA1 | 407.07 | 2.28 | 5.84E−07 | 381.85 | −0.99 | 4.57E−02 |

Hypoxia and Aging

| | | | | | | |
|---|---|---|---|---|---|---|
| CAMK4 | 1155.51 | 1.59 | 5.09E−21 | 455.49 | −1.05 | 2.27E−02 |

DNA Repair and Cancer

| | | | | | | |
|---|---|---|---|---|---|---|
| FOXM1 | 112.43 | 2.05 | 7.92E−05 | 230.91 | 2.23 | 2.23E−06 |

Chronic obstructive pulmonary disease (COPD)

| | | | | | | |
|---|---|---|---|---|---|---|
| HMOX1 | 178.64 | −1.07 | 2.11E−02 | X | X | X |

Inflammatory Bowel Disease (IBD) and Aging

| | | | | | | |
|---|---|---|---|---|---|---|
| IAH1 | 183.87 | 2.99 | 1.37E−07 | 376.60 | −0.82 | 3.29E−02 |

Hearing Loss

| | | | | | | |
|---|---|---|---|---|---|---|
| USP53 | 382.30 | −1.87 | 9.48E−03 | X | X | X |

Renal Failure

| | | | | | | |
|---|---|---|---|---|---|---|
| PTPRO | 856.18 | 2.77 | 2.16E−13 | 851.54 | −1.69 | 2.33E−04 |

### Table 5B. Shared Novel Progeroid Syndromes and AD/ADRD Aging Biomarkers

Expression of differentially expressed genes in PG-NNOP and AD-NNOP-ApoE neural organoids compared to Normal-NNOP in culture for 12 weeks. RNA extraction and gene expression analysis performed by Ampliseq™ (Thermofisher). The AD-NNOP-ApoE were derived from AD patient donors and the iPSCs obtained from the Coreill Biorepository (NJ, USA). Log 2 fold change with a positive value indicates a decrease in expression of an AD-NNOP-ApoE or PG-NNOP gene when compared to Normal-NNOP and with a negative number an increase in gene expression.

| | W12 PG- | W12 AD ApoE-NNOP + | | |
|---|---|---|---|---|
| | NNOP | CGS-21680 Efficacy | | |
| | Log2 Fold | Base | Log2 Fold | |
| Genes | Change | Mean | Change | P-Value |

DNA Stability

| | | | | |
|---|---|---|---|---|
| POGZ | 0.49 | 3020.50 | −0.41 | 0.04320235 |
| POLE3 | −1.18 | 802.06 | 0.86 | 0.0010211 |
| XPA | −1.48 | 768.27 | −0.73 | 0.00119195 |

Telomer Attrition

| | | | | |
|---|---|---|---|---|
| TERF2IP | −0.35 | 6479.14 | 0.59 | 0.00248135 |
| USP36 | −1.51 | 2164.23 | 0.53 | 0.01965268 |

Epigenetic Alterations

| | | | | |
|---|---|---|---|---|
| EZH2 | 0.43 | 918.95 | −0.59 | 0.00601664 |
| HDAC2 | 0.68 | 6051.77 | −0.74 | 0.00024289 |
| KAT6B | 1.27 | 462.53 | −1.13 | 2.8467E−05 |
| KDM3A | −0.68 | 6085.96 | 1.08 | 2.0779E−06 |

Loss of Proteostasis

| | | | | |
|---|---|---|---|---|
| DNAJB2 | −0.61 | 429.34 | 0.96 | 0.00067327 |
| DNAJC1 | −1.16 | 3062.56 | −0.47 | 0.04725516 |
| DNAJC6 | 1.55 | 1541.28 | 0.57 | 0.00972534 |
| USP31 | 1.15 | 448.99 | −0.56 | 0.0499356 |
| VCP | −0.55 | 7173.06 | 0.55 | 0.02692127 |

Deregulated Nutrient Sensing

| | | | | |
|---|---|---|---|---|
| CERS6 | 1.03 | 1577.13 | −0.88 | 1.0499E−05 |
| MCHR1 | 2.43 | 125.07 | −1.38 | 0.00084734 |
| SESN2 | −0.41 | 1348.89 | 1.43 | 2.2927E−11 |

Cellular Senescence

| | | | | |
|---|---|---|---|---|
| CDK1 | 0.84 | 485.01 | −1.33 | 1.3518E−06 |
| CDKN1A | −2.56 | 1777.98 | 1.10 | 3.5106E−08 |
| LMNB2 | 1.28 | 448.98 | 0.54 | 0.04396267 |

Stem Cell Exhaustion

| | | | | |
|---|---|---|---|---|
| SLC7A1 | 0.8 | 784.77 | −1.32 | 5.2415E−08 |

Mitochondrial Dysfunction

| | | | | |
|---|---|---|---|---|
| TOMM40 | −0.95 | 442.03 | 0.57 | 0.04152486 |

Altered Intercellular Communication

| | | | | |
|---|---|---|---|---|
| ALDOC | −1.28 | 1041.94 | 1.48 | 1.7445E−11 |
| MT2A | −3.20 | 338.57 | −0.63 | 0.03337575 |
| SLC2A3 | −0.91 | 16892.73 | 0.66 | 0.00046456 |
| SLC35F1 | 1.15 | 512.61 | −1.07 | 7.3586E−05 |
| SLC38A1 | 1.65 | 1011.95 | −0.66 | 0.00196691 |

Disabled Macrophagy

| | | | | |
|---|---|---|---|---|
| SQSTM1 | −0.57 | 3293.24 | 0.54 | 0.00632466 |

Chronic Inflammation

| | | | | |
|---|---|---|---|---|
| NLRC5 | 2.64 | 26.14 | −2.29 | 0.00406829 |

Dysbiosis

| | | | | |
|---|---|---|---|---|
| NAPEPLD | 0.79 | 454.61 | −0.60 | 0.01441121 |
| TLR4 | 4.18 | 7.49 | −3.30 | 0.03212542 |

Immunity

| | | | | |
|---|---|---|---|---|
| ID2 | 2.37 | 665.65 | 0.70 | 0.00287036 |
| IDI1 | −1.61 | 1343.42 | 0.49 | 0.01529484 |

Progeroid syndromes (Accelerated Aging)

| | | | | |
|---|---|---|---|---|
| WRN | −0.85 | X | X | X |
| DHX9 | 1.22 | 963.76 | −0.75 | 0.00041374 |

Osteoarthritis

| | | | | |
|---|---|---|---|---|
| SPP1 | 2.59 | 164.84 | −1.09 | 6.37E−03 |

Migraine

| | | | | |
|---|---|---|---|---|
| ZEB2 | 2.08 | 1322.87 | −0.82 | 9.14E−03 |
| PLCE1 | 1.5 | 151.27 | −0.87 | 1.43E−02 |

Atherosclerosis

| | | | | |
|---|---|---|---|---|
| ANGPT1 | 1.98 | 44.16 | −1.33 | 2.8E−02 |

Ceramides and Aging

| | | | | |
|---|---|---|---|---|
| SPTSSA | −0.83 | 907.52 | 0.49 | 4.34E−02 |
| PLEKHA8 | 0.75 | 704.44 | 0.48 | 3.81E−02 |

Neuropathic Pain

| | | | | |
|---|---|---|---|---|
| AK2 | −0.93 | 952.53 | 0.84 | 1.42E−04 |

Inherited Cancer Syndrome

| | | | | |
|---|---|---|---|---|
| DHFR | 1.15 | 383.79 | −1.13 | 1.08E−04 |

Heart Disease

| | | | | |
|---|---|---|---|---|
| BHLHE40 | −1.51 | 5561.37 | 1.4 | 1.46E−02 |
| ABCA | 2.28 | 469.62 | 0.73 | 3.12E−03 |

Hypoxia and Aging

| | | | | |
|---|---|---|---|---|
| CAMK4 | 1.59 | 1063.92 | −0.75 | 7.84E−04 |

DNA Repair and Cancer

| | | | | |
|---|---|---|---|---|
| FOXM1 | 2.05 | 73.20 | −1.19 | 1.28E−02 |

Chronic obstructive pulmonary disease (COPD)

| | | | | |
|---|---|---|---|---|
| HMOX1 | −1.07 | 388.55 | 0.99 | 5.44E−03 |

Inflammatory Bowel Disease (IBD) and Aging

| | | | | |
|---|---|---|---|---|
| IAH1 | 2.99 | 223.26 | −0.71 | 4.96E−02 |

Hearing Loss

| | | | | |
|---|---|---|---|---|
| USP53 | −1.87 | 513.82 | 0.62 | 2.56E−02 |

Renal Failure

| | | | | |
|---|---|---|---|---|
| PTPRO | 2.77 | 559.85 | −0.62 | 1.45E−02 |

### Table 6: Significant Therapeutic Reversal of Shared Novel Progeroid Syndromes and AD/ADRD Aging Biomarkers by Adenosine 2A Receptor Agonist CGS-21680.

Expression of differentially expressed genes in PG-NNOP neural organoids compared to Normal-NNOP in culture for 12 weeks. Expression of differentially expressed genes in AD-NNOP-ApoE neural organoids with CGS-21680 treatment compared to AD-NNOP-ApoE without drug treatment in culture for 12 weeks. AD-NOP-ApoE neural organoids for >6 weeks in culture were used to test the efficacy of the highly selective A2A Receptor agonist CGS-21680. CGS-21680 was tested at 1microM; A2A receptor EC50 of 110 nM). AD-NOP were incubated with vehicle (0.1% DMSO; control) or CGS-21680 dissolved in DMSO at 1microM for 4 days and harvested for RNA extraction and gene expression analysis by Ampliseq™ (Thermofisher). Log 2 fold change with a positive value indicates a decrease in expression of a PG-NNOP gene when compared to Normal-NNOP or a AD-NNOP-ApoE gene with CGS-21680 compared to AD-NNOP-ApoE without the drug treatment, and with a negative number indicates an increase in gene expression. X shows that the gene expression was not significantly altered. The log 2 fold change numbers in bold show a complete reversal, or a reduction in magnitude of change from the log 2 fold change in PG-NNOP without drug treatment (in italic); suggesting that the drug is capable of correcting the dysregulated gene expression in PG-NNOP towards normalcy.

| | W12 PG-NNOP | W12 PG-NNOP + FAAH-I | | |
|---|---|---|---|---|
| | Log2 Fold | Base | Log2 Fold | |
| Genes | Change | Mean | Change | P-Value |

DNA Stability

| | | | |
|---|---|---|---|
| POGZ | 0.49 | X | X |
| POLE3 | −1.18 | X | X |
| XPA | −1.48 | X | X |

Telomer Attrition

| | | | |
|---|---|---|---|
| TERF2IP | −0.35 | X | X |
| USP36 | −1.51 | X | X |

Epigenetic Alterations

| | | | | |
|---|---|---|---|---|
| EZH2 | 0.43 | 894.52 | −0.5 | 0.0016266 |
| HDAC2 | 0.68 | X | X | |
| KAT6B | 1.27 | X | X | |
| KDM3A | −0.68 | X | X | |

Loss of Proteostasis

| | | | | |
|---|---|---|---|---|
| DNAJB2 | −0.61 | X | X | |
| DNAJC1 | −1.16 | X | X | |
| DNAJC6 | 1.55 | X | X | |
| USP31 | 1.15 | 353.83 | 0.62 | 0.012553 |
| VCP | −0.55 | X | X | |

Deregulated Nutrient Sensing

| | | | |
|---|---|---|---|
| CERS6 | 1.03 | X | X |
| MCHR1 | 2.43 | X | X |
| SESN2 | −0.41 | X | X |
| SLC43A2 | −1.1 | X | X |

Cellular Senescence

| | | | | |
|---|---|---|---|---|
| CDK1 | 0.84 | X | X | |
| CDKN1A | −2.56 | 5486.19 | 0.77 | 2.221E−06 |
| LMNB2 | 1.28 | 859.52 | −0.67 | 4.304E−05 |

Stem Cell Exhaustion

| | | | |
|---|---|---|---|
| SLC7A1 | 0.8 | X | X |

Mitochondrial Dysfunction

| | | | |
|---|---|---|---|
| TOMM40 | −0.95 | X | X |

Altered Intercellular Communication

| | | | | |
|---|---|---|---|---|
| ALDOC | −1.28 | X | X | |
| MT2A | −3.20 | 970.60 | 2.77 | 3.992E−34 |
| SLC2A3 | −0.91 | X | X | |
| SLC35F1 | 1.15 | 511.20 | −0.65 | 0.0211513 |
| SLC38A1 | 1.65 | 368.37 | −0.57 | 0.0490046 |

Disabled Macrophagy

| | | | |
|---|---|---|---|
| SQSTM1 | −0.57 | X | X |

Chronic Inflammation

| | | | |
|---|---|---|---|
| NLRC5 | 2.64 | X | X |

Dysbiosis

| | | | |
|---|---|---|---|
| NAPEPLD | 0.79 | X | X |
| TLR4 | 4.18 | X | X |

Immunity

| | | | | |
|---|---|---|---|---|
| ID2 | 2.37 | 1311.62 | 0.35 | 0.0244772 |
| IDI1 | −1.61 | 2568.30 | 1.15 | 1.861E−06 |

Progeroid syndromes (Accelerated Aging)

| | | | |
|---|---|---|---|
| WRN | −0.85 | X | X |
| DHX9 | 1.22 | X | X |

### Table 7: Partial Therapeutic Reversal of Shared Novel Progeroid Syndromes and AD/ADRD Aging Biomarkers by Fatty Acid Amide Hydrolase Inhibitor (FAAH-1).

Expression of differentially expressed genes in PG-NNOP neural organoids compared to Normal-NNOP in culture for 12 weeks is shown, as well as expression of differentially expressed genes in PG-NNOP neural organoids with PF-3845 treatment compared to PG-NNOP without drug treatment in culture for 12 weeks. PG-NNOP neural organoids for >12 weeks in culture were used to test the efficacy of the highly selective Fatty Acid Amide Hydroxylase (FAAH) inhibitor PF-3845 was tested at 10microM). PG-NNOP neural organoids were incubated with vehicle (0.1% DMSO; control) or PF-3845 dissolved in DMSO at 10microM for 4 days and harvested for RNA extraction and gene expression analysis by Ampliseq™ (Thermofisher). Log 2 fold change with a positive value indicates a decrease in expression of a PG-NNOP gene when compared to Normal-NNOP or a PG-NNOP gene with FAAH-I compared to PG-NNOP without the drug treatment, and with a negative number indicates an increase in gene expression. X shows that the gene expression was not significantly altered. The log 2 fold change numbers in bold show a complete reversal, or a reduction in magnitude of change from the log 2 fold change in PG-NNOP without drug treatment (in italic); suggesting that the drug is capable of correcting the dysregulated gene expression in PG-NNOP towards normalcy.

| | | W12 AG PG-NNOP + | | |
|---|---|---|---|---|
| | W12 PG-NNOP | Metformin | | |
| | Log2 Fold | Base | Log2 Fold | |
| Genes | Change | Mean | Change | P-Value |

DNA Stability

| | | | | |
|---|---|---|---|---|
| POGZ | 0.49 | 1815.20 | 0.36 | 0.0379718 |
| POLE3 | −1.18 | 888.84 | −0.25 | 0.0325403 |
| XPA | −1.48 | X | X | |

Telomer Attrition

| | | | |
|---|---|---|---|
| TERF2IP | −0.35 | X | X |
| USP36 | −1.51 | X | X |

Epigenetic Alterations

| | | | |
|---|---|---|---|
| EZH2 | 0.43 | X | X |
| HDAC2 | 0.68 | X | X |
| KAT6B | 1.27 | X | X |
| KDM3A | −0.68 | X | X |

Loss of Proteostasis

| | | | | |
|---|---|---|---|---|
| DNAJB2 | −0.61 | X | X | |
| DNAJC1 | −1.16 | 1335.80 | −0.26 | 0.0266212 |
| DNAJC6 | 1.55 | X | X | |
| USP31 | 1.15 | 359.95 | 0.67 | 0.0037141 |
| VCP | −0.55 | 5337.41 | 0.33 | 0.0076035 |

Deregulated Nutrient Sensing

| | | | |
|---|---|---|---|
| CERS6 | 1.03 | X | X |
| MCHR1 | 2.43 | X | X |
| SESN2 | −0.41 | X | X |

Cellular Senescence

| | | | | |
|---|---|---|---|---|
| CDK1 | 0.84 | 456.38 | 0.52 | 0.02425 |
| CDKN1A | −2.56 | 3589.50 | −0.35 | 0.0342799 |
| LMNB2 | 1.28 | 917.67 | −0.42 | 0.0016609 |

Stem Cell Exhaustion

| | | | |
|---|---|---|---|
| SLC7A1 | 0.8 | X | X |

Mitochondrial Dysfunction

| | | | |
|---|---|---|---|
| TOMM40 | −0.95 | X | X |

Altered Intercellular Communication

| | | | | |
|---|---|---|---|---|
| ALDOC | −1.28 | X | X | |
| MT2A | −3.20 | 639.99 | 2.06 | 5.331E−30 |
| SLC2A3 | −0.91 | X | X | |
| SLC35F1 | 1.15 | 534.83 | −0.47 | 0.0260689 |
| SLC38A1 | 1.65 | X | X | |

Disabled Macrophagy

| | | | |
|---|---|---|---|
| SQSTM1 | −0.57 | X | X |

Chronic Inflammation

| | | | |
|---|---|---|---|
| NLRC5 | 2.64 | X | X |

Dysbiosis

| | | | |
|---|---|---|---|
| NAPEPLD | 0.79 | X | X |
| TLR4 | 4.18 | X | X |

Immunity

| | | | | |
|---|---|---|---|---|
| ID2 | 2.37 | 1615.01 | 0.86 | 1.379E−06 |
| IDI1 | −1.61 | X | X | |

Werner Syndrome (Accelerated Aging)

| | | | | |
|---|---|---|---|---|
| WRN | −0.85 | 337.54 | 0.71 | 0.0008551 |
| DHX9 | 1.22 | X | X | |

### Table 8: Partial Therapeutic Reversal of Shared Novel Progeroid Syndromes and AD/ADRD Aging Biomarkers by Metformin.

Expression of differentially expressed genes in PG-NNOP neural organoids compared to Normal-NNOP in culture for 12 weeks and expression of differentially expressed genes in PG-NNOP neural organoids with metformin treatment compared to PG-NNOP without drug treatment in culture for 12 weeks is shown. PG-NNOP neural organoids for >12 weeks in culture were used to test the efficacy of the highly selective Metformin/was tested at 0.28 mg/ml). PG-NNOP neural organoids were incubated with vehicle (HBBS; control) or metformin dissolved in HBBS at 0.28 mg/ml for 4 days and harvested for RNA extraction and gene expression analysis by Ampliseq™ (Thermofisher). Log 2 fold change with a positive value indicates a decrease in expression of a PG-NNOP gene when compared to Normal-NNOP or a PG-NNOP gene with Metformin compared to PG-NNOP without the drug treatment, and with a negative number indicates an increase in gene expression. X shows that the gene expression was not significantly altered. The log 2 fold change numbers in bold show a complete reversal, or a reduction in magnitude of change from the log 2 fold change in PG-NNOP without drug treatment (in italic); suggesting that the drug is capable of correcting the dysregulated gene expression in PG-NNOP towards normalcy.

| | | W12 PG-NNOP + | | |
|---|---|---|---|---|
| | W12 PG-NNOP | Rapamycin | | |
| | Log2 Fold | Base | Log2 Fold | |
| Genes | Change | Mean | Change | P-Value |

DNA Stability

| | | | | |
|---|---|---|---|---|
| POGZ | 0.49 | X | X | |
| POLE3 | −1.18 | 1102.07 | 0.75 | 0.0014347 |
| XPA | −1.48 | X | X | |

Telomer Attrition

| | | | | |
|---|---|---|---|---|
| TERF2IP | −0.35 | X | X | |
| USP36 | −1.51 | 1261.53 | 0.99 | 0.0006632 |

Epigenetic Alterations

| | | | |
|---|---|---|---|
| EZH2 | 0.43 | X | X |
| HDAC2 | 0.68 | X | X |
| KAT6B | 1.27 | X | X |
| KDM3A | −0.68 | X | X |

Loss of Proteostasis

| | | | |
|---|---|---|---|
| DNAJB2 | −0.61 | X | X |
| DNAJC1 | −1.16 | X | X |
| DNAJC6 | 1.55 | X | X |
| USP31 | 1.15 | X | X |
| VCP | −0.55 | X | X |

Deregulated Nutrient Sensing

| | | | |
|---|---|---|---|
| CERS6 | 1.03 | X | X |
| MCHR1 | 2.43 | X | X |
| SESN2 | −0.41 | X | X |
| SLC43A2 | −1.1 | X | X |

Cellular Senescence

| | | | | |
|---|---|---|---|---|
| CDK1 | 0.84 | X | X | |
| CDKN1A | −2.56 | 5689.18 | 1.21 | 0.0133519 |
| LMNB2 | 1.28 | 676.83 | −0.96 | 0.001755 |

Stem Cell Exhaustion

| | | | |
|---|---|---|---|
| SLC7A1 | 0.8 | X | X |

Mitochondrial Dysfunction

| | | | |
|---|---|---|---|
| TOMM40 | −0.95 | X | X |

Altered Intercellular Communication

| | | | | |
|---|---|---|---|---|
| ALDOC | −1.28 | X | X | |
| MT2A | −3.20 | 1095.49 | 3.23 | 5.191E−34 |
| SLC2A3 | −0.91 | X | X | |
| SLC35F1 | 1.15 | 414.19 | −0.83 | 0.0341744 |
| SLC38A1 | 1.65 | 270.50 | −1.15 | 0.0090265 |

Disabled Macrophagy

| | | | |
|---|---|---|---|
| SQSTM1 | −0.57 | X | X |

Chronic Inflammation

| | | | |
|---|---|---|---|
| NLRC5 | 2.64 | X | X |

Dysbiosis

| | | | |
|---|---|---|---|
| NAPEPLD | 0.79 | X | X |
| TLR4 | 4.18 | X | X |

Immunity

| | | | | |
|---|---|---|---|---|
| ID2 | 2.37 | X | X | |
| IDI1 | −1.61 | 2569.05 | 1.48 | 1.789E−06 |

Werner Syndrome (Accelerated Aging)

| | | | |
|---|---|---|---|
| WRN | −0.85 | X | X |
| DHX9 | 1.22 | X | X |

### Table 9: Partial Therapeutic Reversal of Shared Novel Progeroid Syndromes and AD/ADRD Aging Biomarkers by Rapamycin.

Expression of differentially expressed genes in PG-NNOP neural organoids compared to Normal-NNOP in culture for 12 weeks and expression of differentially expressed genes in PG-NNOP neural organoids with rapamycin treatment compared to PG-NNOP without drug treatment in culture for 12 weeks is shown. PG-NNOP neural organoids for >12 weeks in culture were used to test the efficacy of the highly selective Rapamycin was tested at 50 nM). PG-NNOP neural organoids were incubated with vehicle (HBBS; control) or metformin dissolved in HBBS at 50 nM for 4 days and harvested for RNA extraction and gene expression analysis by Ampliseq™ (Thermofisher). Log 2 fold change with a positive value indicates a decrease in expression of an PG-NNOP gene when compared to Normal-NNOP and with a negative number an increase in gene expression. X shows that the gene expression was not significantly altered. The log 2 fold change numbers in bold show a complete reversal, or a reduction in magnitude of change from the log 2 fold change in PG-NNOP without drug treatment (in italic); suggesting that the drug is capable of correcting the dysregulated gene expression in PG-NNOP towards normalcy.

Aging Biomarkers in Fibroblast

| | Genes |
|---|---|
| Functions | |
| DNA stability | POLE4 |
| Telomer Attrition | X |
| | X |
| Epigenetic Alterations | HIST1H3D |
| | KDM2B |
| | SMARCA5 |
| Loss of Proteostasis | VCP |
| Deregulated Nutrient Sensing | COASY |
| | SESN2 |
| | SLC38A7 |
| | SLC3A2 |
| | PTPRG |
| | SELENBP1 |
| Cellular Senescence | IDI1 |
| Stem Cell Exhaustion | SLC7A1 |
| | SLC27A3 |
| Mitochondrial Dysfunction | SLC25A4 |
| | SDHC |
| | PINK1 |
| Altered Intercellular Communication | ALDOC |
| | ARHGAP35 |
| | SLC2A3 |
| | SLC31A1 |
| Disabled Macrophagy | X |
| Chronic Inflammation | FURIN |
| Dysbiosis | TLR4 |
| Immunity | LGMN |
| | XBP1 |
| | DDX17 |
| | XBP1 |
| Progeroid syndromes (Accelerated Aging) | WRN |
| Clinical Features | |
| Cancer | ATF3 |
| Sleep | BHLHE40 |
| Fibrosis | DOK5 |
| Leukoencephalopathy | EIF1 |
| Schizophrenia and bipolar | PDE4B |
| Hypertension | PTGIS |
| Pain | SCN1A |
| Memory | SCN2A |
| Vision | SLC17A7 |
| Epileptic Encephalopathy | SLC1A2 |
| Cystinuria | SLC1A5 |
| Hyperinsulinemic Hypoglycemia | SLC25A36 |
| Hypomagnesemia 1 | SLC41A3 |
| Alzheimer Disease 9 | SORL1 |

### Table 10: Diagnostics Shared Novel Progeroid Syndromes and AD/ADRD Aging Biomarkers Expressed in Fibroblasts

Expression of differentially expressed genes from three AD-NNOP fibroblasts AD-PSEN2; AD-SPOR; and AD-ApoE derived from AD patient donors compared to Normal fibroblasts in culture and then compared to those of PG-NNOP neural organoids identifies aging specific biomarkers is shown. RNA extraction and gene expression analysis performed by Ampliseq™ (Thermofisher). Some biomarkers of recognizable clinical diseases of features associated with aging are also included. X shows that the gene expression was not significantly altered.

| | | |
|---|---|---|
| base | log2 Fold | |
| Mean | Change | p-value |

Endocannabioid Anandamide Receptor
CNR1

| | | | |
|---|---|---|---|
| AD-APP | 638.69 | 2.77 | 7.547E−12 |
| AD-PSEN2 | 498.28 | 7.05 | 1.74E−36 |
| AD-SPOR | 431.50 | 3.93 | 0.0019011 |
| AD-ApoE | 619.34 | 2.20 | 2.176E−21 |

Adenosine Receptors
ADORA2A

| | | | |
|---|---|---|---|
| AD-APP | | | |
| AD-PSEN2 | 37.83 | 2.21 | 0.0270773 |
| AD-SPOR | 30.95 | 2.17 | 0.031057 |
| AD-ApoE | 619.34 | 2.20 | 2.176E−21 |

ADORA1

| | | | |
|---|---|---|---|
| AD-APP | 150.83 | 1.94 | 0.0107792 |
| AD-PSEN2 | 135.05 | 2.19 | 1.111E−05 |
| AD-SPOR | 104.32 | 2.77 | 0.0002154 |
| AD-ApoE | 141.79 | 2.06 | 3.47E−05 |

ADORA2B

| | | | |
|---|---|---|---|
| AD-APP | | | |
| AD-PSEN2 | 213.46 | −1.17 | 0.0423331 |
| AD-SPOR | 270.49 | −2.02 | 0.0423331 |
| AD-ApoE | | | |

Sphingosine-1-phosphate receptor
S1PR1

| | | | |
|---|---|---|---|
| AD-APP | | | |
| AD-PSEN2 | 396.32 | −3.12 | 1.012E−15 |
| AD-SPOR | 305.92 | −3.02 | 0.0060121 |
| AD-ApoE | | | |

Table 11. Key Therapeutic Biomarkers Dysregulated in AD-NNOP Models. Expression of differentially expressed genes in neural organoids of several AD models compared to Normal-NNOP is shown.

| | W12 PG-NNOP | | | W12 AD AD-NNOP-ApoE | | |
|---|---|---|---|---|---|---|
| | | Log2 | | | Log2 | |
| | Base | Fold | | Base | Fold | |
| Genes | Mean | Change | P-Value | Mean | Change | P-Value |

Environmental factors

| | | | | | | |
|---|---|---|---|---|---|---|
| MT2A | 1533.21 | −3.20 | 7.19E−13 | X | X | X |
| SLC7A5 | | | | 1199.91 | 2.20 | 4.36E−11 |

Nutritional factors

| | | | | | | |
|---|---|---|---|---|---|---|
| SELENBP1 | 74.51 | 1.74 | 3.20E−02 | X | X | X |
| RARB | X | X | X | 612.34 | 1.60 | 1.52E−03 |

Table 12. Shared Novel Progeroid syndromes and AD/ADRD Biomarkers involved in nutritional uptake and susceptibility to environmental toxins. Expression of differentially expressed genes in PG-NNOP and AD-NNOP-ApoE neural organoids compared to Normal-NNOP in culture for 12 weeks is shown. RNA extraction and gene expression analysis performed by Ampliseq™ (Thermofisher). The AD-NNOP-ApoE were derived from AD patient donors and the iPSCs obtained from the Coreill Biorepository (NJ, USA). Log 2 fold change with a positive value indicates a decrease in expression of an AD-NNOP-ApoE or PG-NNOP gene when compared to Normal-NNOP and with a negative number an increase in gene expression.

| | | W12 AD ApoE-NNOP + | | |
|---|---|---|---|---|
| | W12 PG-NNOP | CGS-21680 Efficacy | | |
| | Log2 Fold | Base | Log2 Fold | |
| Genes | Change | Mean | Change | P-Value |

Environmental Factors

| | | | | |
|---|---|---|---|---|
| MT2A | −3.20 | 338.57 | −0.63 | 3.34E−02 |
| SLC7A5 | X | 1003.79 | 0.52 | 2.94E−02 |

Nutritional Factors

| | | | | |
|---|---|---|---|---|
| SELENBP1 | 1.74 | 48.05 | −1.15 | 4.57E−02 |
| RARB | X | 187.68 | −0.87 | 1.77E−02 |

Table 13: Significant Therapeutic Reversal of Shared Novel Progeroid syndromes and AD/ADRD Biomarkers involved in nutritional uptake and susceptibility to environmental toxins by Adenosine 2A Receptor Agonist CGS-21680. Expression of differentially expressed genes in PG-NNOP neural organoids compared to Normal-NNOP in culture for 12 weeks and expression of differentially expressed genes in AD-NNOP-ApoE neural organoids with CGS-21680 treatment compared to AD-NNOP-ApoE without drug treatment in culture for 12 weeks is shown. AD-NNOP-ApoE neural organoids for >6 weeks in culture were used to test the efficacy of the highly selective A2A Receptor agonist CGS-21680. CGS-21680 was tested at 1microM; A2A receptor EC50 of 110 nM). AD-NOP were incubated with vehicle (0.1% DMSO; control) or CGS-21680 dissolved in DMSO at 1microM for 4 days and harvested for RNA extraction and gene expression analysis by Ampliseq™ (Thermofisher). Log 2 fold change with a positive value indicates a decrease in expression of a PG-NNOP gene when compared to Normal-NNOP or a AD-NNOP-ApoE gene with CGS-21680 compared to AD-NNOP-ApoE without the drug treatment, and with a negative number indicates an increase in gene expression. X shows that the gene expression was not significantly altered. The log 2 fold change numbers in bold show a complete reversal, or a reduction in magnitude of change from the log 2 fold change in PG-NNOP without drug treatment (in italic); suggesting that the drug is capable of correcting the dysregulated gene expression in PG-NNOP towards normalcy.

### OTHER EMBODIMENTS

From the foregoing description, it will be apparent that variations and modifications can be made to the invention described herein to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

Having described the invention in detail and by reference to specific aspects and/or embodiments thereof, it will be apparent that modifications and variations are possible without departing from the scope of the invention defined in the appended claims. More specifically, although some aspects of the present invention may be identified herein as particularly advantageous, it is contemplated that the present invention is not limited to these particular aspects of the invention. Percentages disclosed herein can vary in amount by ±10, 20, or 30% from values disclosed and remain within the scope of the contemplated invention.

What is claimed is:

1. A method for reducing or ameliorating disease severity in an individual for a disease, disorder, or disability as a consequence of biological aging comprising administering to the individual a therapeutically effective amount of a pharmaceutical composition comprising a drug capable of reducing or correcting dysfunctional expression of genes related to said disease or disability as a consequence of biological aging in vitro in a neural organoid platform and reducing or ameliorating disease severity in an individual thereby.

2. The method of claim 1, wherein the disease, disorder, or disability is Alzheimer's disease, Alzheimer's Disease Related Dementia (ADRD), Mixed Etiology Dementia (MED), Lewy Body dementia, fronto-temporal dementia, Progeroid syndromes, Hutchinson-Gilford progeroid syndrome, Werner syndrome, vascular contributors to dementia, or cancer.

3. The method of claim 1, wherein the genes related to a disease, disorder, or disability as a consequence of biological aging for which the drug is capable of reducing or correcting dysfunctional expression are set forth in Tables 3-11.

4. The method of claim 1, wherein the neural organoid sample is procured from minutes to hours up to 15 weeks post inducement.

5. The method of claims 1, 2, or 3, wherein the drug is an agonist of an adenosine A2a receptor. (Genbank Accession No. NP_001265429.1)

6. The method of claim 5, wherein the adenosine A2a receptor is encoded by an ADORA2A gene (Genbank Accession No. NM_001278497.2).

7. The method of claim 6, wherein the drug is methyl (1R,4r)-4-(3-(6-amino-9-((2R,3R,4S,5S)-5-(ethylcarbamoyl)-3,4-dihydroxytetrahydrofuran-2-yl)-9H-purin-2-yl)prop-2-yn-1-yl)cyclohexane-1-carboxylate (apadenoson), metformin, or rapamycin.

8. The method of claim 7, wherein the drug is 2-p-(2-Carboxyethyl) phenethylamino-5′-N-ethylcarboxamidoadenosine hydrochloride hydrate (CGS-21680).

9. The method of claims 1, 2, or 3, wherein the drug increases endocannabinoid anandamide receptor activity.

10. The method of claim 9, wherein the endocannabinoid anandamide receptor is encoded by a CNR1 gene (Genbank Accession No. NM_016083.6).

11. The method of claim 10, wherein the drug is an inhibitor of Fatty Acid Amide Hydrolase.

12. The method of claim 11, wherein the drug is PF-3845.

13. A pharmaceutical composition of a drug capable of reducing or ameliorating disease severity in an individual having a disease, disorder, or disability as a consequence of biological aging comprising a therapeutically effective amount of the pharmaceutical composition comprising a drug capable of reducing or correcting dysfunctional expression of genes in vitro in a neural organoid platform that reduces or ameliorates disease severity in an individual thereby.

14. The pharmaceutical composition of claim 13, wherein the disease, disorder, or disability is Alzheimer's disease, Alzheimer's Disease Related Dementia (ADRD), Mixed Etiology Dementia (MED), Lewy Body dementia, fronto-temporal dementia, Progeroid syndromes, Hutchinson-Gilford progeroid syndrome, Werner syndrome, vascular contributors to dementia, or cancer.

15. The pharmaceutical composition of claim 13 or 14, wherein the genes related to a disease, disorder, or disability as a consequence of biological aging for which the drug is capable of reducing or correcting dysfunctional expression are set forth in Tables 3-11.

16. The pharmaceutical composition of claim 15, wherein the drug is an agonist of an adenosine A2a receptor. (Genbank Accession No. NP_001265429.1)

17. The pharmaceutical composition of claim 16, wherein the adenosine A2a receptor is encoded by an ADORA2A gene (Genbank Accession No. NM_001278497.2).

18. The pharmaceutical composition of claim 17, wherein the drug is methyl (1R,4r)-4-(3-(6-amino-9-((2R,3R,4S,5S)-5-(ethylcarbamoyl)-3,4-dihydroxytetrahydrofuran-2-yl)-9H-purin-2-yl)prop-2-yn-1-yl)cyclohexane-1-carboxylate (apadenoson), metformin, or rapamycin.

19. The pharmaceutical composition of claim 17, wherein the drug is 2-p-(2-Carboxyethyl) phenethylamino-5′-N-ethylcarboxamidoadenosine hydrochloride hydrate (CGS-21680).

20. The pharmaceutical composition of claim 15, wherein the drug increases endocannabinoid anandamide receptor activity.

21. The pharmaceutical composition of claim, wherein the endocannabinoid anandamide receptor is encoded by a CNR1 gene (Genbank Accession No. NM_016083.6).

22. The pharmaceutical composition of claim 21, wherein the drug is an inhibitor of Fatty Acid Amide Hydrolase.

23. The pharmaceutical composition of claim 22, wherein the drug is PF-3845.

24. A method for performing gene therapy in a cell to correct expression of one or a plurality of adenosine receptor network components identified as ADORA1, ADORA2A, ADORA2B and CNR1, and EZH2 genes and their modulators resulting in modification of gene expression in the one or a plurality of components.

25. The method of claim 24, wherein gene therapy is performed using long non-coding RNA species.

26. The method of claim 24, wherein gene therapy is performed using an anti-sense oligonucleotide (ASO) to modify expression of the adenosine receptor network components identified as ADORA1, ADORA2A, ADORA2B, CNR1 and EZH2.

27. The method of claim 26, wherein gene therapy effects are measured by detecting changes in expression of genes encoding human LMNA, WRN, DHX9, A2M, PSEN2, ApoE, APP variants or sporadic AD.

28. The method of claim 24, wherein gene therapy is performed using an anti-sense oligonucleotide (ASO) to correct expression of the human LMNA, WRN, DHX9, A2M, PSEN2, ApoE, APP variants or sporadic AD.

29. The method of claim 27, wherein the measured genes comprise one or a plurality of genes as identified in Tables 3-11.

30. The method of claim 26, wherein the method is used for diagnostic, therapeutic target discovery and drug action discovery for aging including dementia, Alzheimer's disease, Progeroid syndromes, and aging related comorbidities as listed in Table 10 and Table 5B, and cancer.

31. The method of claim 1, wherein data obtained from neural organoid data is corroborated in postmortem tissues from idiopathic patients and identifies changes in gene expression for aging including age-related dementia and cancers.

32. The method of claim 31, wherein the method can be used with induced pluripotent stem cells from any skin cell, tissue, or organ from the human body for diagnostics, therapeutic target discovery, and drug development.

33. The method of claims 1 or 24, wherein the method and/or neural organoid is used in patient specific toxicology to identify genes involved in a patient's selective vulnerability to infectious agents or environmental toxins.

34. The method of claim 1 or 24, wherein the method can be used to identify nutritional and toxicological care that can begin even before birth so that the child develops normally in utero.

35. The method of claims 1 or 24, wherein the measured genes are representative of proteins or their metabolites dysregulated in disease, disorder, or disability.

36. The method of any one of claims 1 or 24, wherein the method can be used to diagnosis of aging including age-related dementia and cancers at birth.

37. A method for treating aging including age-related dementia and cancers in a human, using a patient-specific pharmacotherapy, the method comprising:
a) procuring one or a plurality of cell samples from a patient, comprising one or a plurality of cell types including but not limited to fibroblasts, buccal cavity cells, or peripheral blood monocytes;
b) detecting genes from the procured one or a plurality of cell samples that are differentially expressed, wherein these genes are also differentially expressed in humans with accelerated aging including age-related dementia and cancers;
c) preparing a neural organoid from the procured one or a plurality of cell samples and detecting additional genes that are differentially expressed compared to neural organoid obtained from a healthy patient;
d) performing assays on the neural organoid from the procured one or a plurality of cell samples to identify therapeutic agents that alter one or more of the differentially expressed genes detected in b) and c); and
d) administering one or more of the therapeutic agents for aging including age-related dementia and cancers to treat the patient.

38. The method of claim 37, wherein the fibroblast is used as a developmental tool for predicting the risk of aging including age-related dementia and cancers in a newborn.

39. The method of claim 37, wherein the fibroblasts are used as a drug discovery tool.

40. The method of claim 37, wherein the fibroblasts are used to follow the onset, progression, and/or treatment of aging including age-related dementia and cancers.
